SEQUENCE LISTING (26 sequences)

SEQ ID NO: 1 - DNA, 13 bp, Artificial Sequence
  chemically synthesized
tcgnntcgnntcg

SEQ ID NO: 2 - DNA, 24 bp, Artificial Sequence
  chemically synthesized
tcgtcgttttgtcgttttgtcgtt

SEQ ID NO: 3 - DNA, 22 bp, Artificial Sequence
  chemically synthesized
tgactgtgaacgttcgagatga

SEQ ID NO: 4 - DNA, 40 bp, Artificial Sequence
  chemically synthesized
ccagccacctactccaccagtgccaggactgcttgagggg

SEQ ID NO: 5 - DNA, 40 bp, Artificial Sequence
  chemically synthesized
ctaacgtttaaccaggatcccccaagtccctgctagtggg

SEQ ID NO: 6 - DNA, 40 bp, Artificial Sequence
  chemically synthesized
tgggcgttaccactacaggtccagatttgtctgtccgggg

SEQ ID NO: 7 - DNA, 40 bp, Artificial Sequence
  chemically synthesized
gggatctacggctaaacatctaacgctctttggccctggg

SEQ ID NO: 8 - DNA, 39 bp, Artificial Sequence
  chemically synthesized
cgctcccttatatatccgacgtgactaatactgtggggc

SEQ ID NO: 9 - DNA, 40 bp, Artificial Sequence
  chemically synthesized
gcacataaaactttaccccgacgtggaggacgttcttggc

SEQ ID NO: 10 - DNA, 39 bp, Artificial Sequence
  chemically synthesized
ccagtcgtacaggaaacatgcgttctagatgttcggggc

SEQ ID NO: 11 - DNA, 39 bp, Artificial Sequence
  chemically synthesized
cgcagcgtatggattcagggttggatcgtgtaggggggg

SEQ ID NO: 12 - DNA, 40 bp, Artificial Sequence
  chemically synthesized
cgcgtgaagaaaaggagcagtcataaacgctaatcgtgcc

SEQ ID NO: 13 - DNA, 39 bp, Artificial Sequence
  chemically synthesized
tctgcggggaagagctacgttactagtcgtgtgtccgtg

SEQ ID NO: 14 - DNA, 39 bp, Artificial Sequence
  chemically synthesized
gcggccattcaggaaacgttaatgtcgatctacgttggc

SEQ ID NO: 15 - DNA, 40 bp, Artificial Sequence
  chemically synthesized
cctggctgttccgaaacatatccacagttgttggcccagg

SEQ ID NO: 16 - DNA, 39 bp, Artificial Sequence
  chemically synthesized
tctgtggggaagagctacgttactagtcgtgtgtccgtg

SEQ ID NO: 17 - DNA, 39 bp, Artificial Sequence
  chemically synthesized
tctgcggggaagagctatgttactagtcgtgtgtccgtg

SEQ ID NO: 18 - DNA, 39 bp, Artificial Sequence
  chemically synthesized
tctgcggggaagagctacgttactagttgtgtgtccgtg

SEQ ID NO: 19 - DNA, 39 bp, Artificial Sequence
  chemically synthesized
tctgcggggaagagctacgttactagtcgtgtgtctgtg

SEQ ID NO: 20 - DNA, 39 bp, Artificial Sequence
  chemically synthesized
ccagttgtacaggaaacatgcgttctagatgttcggggc

SEQ ID NO: 21 - DNA, 39 bp, Artificial Sequence
  chemically synthesized
ccagtcgtacaggaaacatgtgttctagatgttcggggc

SEQ ID NO: 22 - DNA, 39 bp, Artificial Sequence
  chemically synthesized
ccagtcgtacaggaaacatgcgttctagatgtttggggc

SEQ ID NO: 23 - DNA, 35 bp, Artificial Sequence
  chemically synthesized
ccagtcgtacaggaaacatgcgttctagatgttcg

SEQ ID NO: 24 - DNA, 35 bp, Artificial Sequence
  chemically synthesized
ccagttgtacaggaaacatgcgttctagatgttcg

SEQ ID NO: 25 - DNA, 32 bp, Artificial Sequence
  chemically synthesized
cgcagcgtatggattcagggttggatcgtgta

SEQ ID NO: 26 - DNA, 20 bp, Artificial Sequence
  chemically synthesized
tccatgacgttcctgacgtt

(19) United States
(12) Patent Application Publication
(10) Pub. No.: US 2005/0175630 A1
(43) Pub. Date: Aug. 11, 2005

(54) Immunogenic compositions and methods of use thereof

(76) Inventors: Eyal Raz, Del Mar, CA (US); Joshua Fierer, LaJolla, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/021,821

(22) Filed: Dec. 22, 2004

Related U.S. Application Data
(60) Provisional application No. 60/564,913, filed on Apr. 22, 2004.
(60) Provisional application No. 60/532,786, filed on Dec. 23, 2003.

(51) Int. Cl. A61K 39/116; A61K 39/02; A61K 39/108; A61K 39/04; A61K 39/095; A61K 39/112

(52) U.S. Cl. 424/203.1; 424/248.1; 424/249.1; 424/258.1; 424/241.1; 424/235.1

(57) ABSTRACT

The present invention provides an immunogenic composition comprising lethally irradiated bacteria formulated for mucosal delivery. The present invention further provides methods of preparing a subject immunogenic composition. The present invention further provides a method of inducing an immune response in an individual to an antigen, the method generally involving administering a subject immunogenic composition to a mucosal tissue of the individual.

### CROSS-REFERENCE

This application claims the benefit of U.S. Provisional Patent Application No. 60/532,786, filed Dec. 23, 2003, and U.S. Provisional Patent Application No. 60/564,913, filed Apr. 22, 2004, which applications are incorporated herein by reference in their entirety.

### STATEMENT REGARDING FEDERALLY SPONSORED RESEARCH

The U.S. government may have certain rights in this invention, pursuant to grant nos. AI40682 and AI47884 awarded by the National Institutes of Health.

### FIELD OF THE INVENTION

The present invention is in the field of immunogenic compositions, and in particular immunogenic compositions comprising irradiated bacteria, formulated for mucosal delivery, for stimulating an immune response to a pathogenic bacterium.

### BACKGROUND OF THE INVENTION

Vaccines currently in use for stimulating immune responses, particularly protective immune responses, to pathogenic bacteria generally include isolated proteins or other macromolecular components of bacteria. Subunit vaccines, a strategy for new vaccine development, are based on the identification, isolation or synthesis, and purification of relevant microbial antigens. These antigens are then co-delivered with an appropriate immune stimulant (adjuvant) to elicit protective immunity. Since identification of individual protective antigens that are highly conserved is difficult, the development of an effective subunit vaccine is slow, expensive and hard to accomplish.

There is a need in the art for improved vaccines that stimulate an immune response to pathogenic bacteria. The present invention addresses this need.

Literature

Malik et al. (1991) Proc. Natl. Acad Sci. USA 88:3300; U.S. Pat. Nos. 6,303,130, 6,264,952, and 6,610,661; U.S. patent publication Nos. 20020164341, 20030139364, and 20030175731.

### SUMMARY OF THE INVENTION

The present invention provides an immunogenic composition comprising lethally irradiated bacteria formulated for mucosal delivery. The present invention further provides methods of preparing a subject immunogenic composition. The present invention further provides a method of inducing an immune response in an individual to an antigen, the method generally involving administering a subject immunogenic composition to a mucosal tissue of the individual.

### BRIEF DESCRIPTION OF THE DRAWINGS

FIGS. 1A and 1B depicts the efficacy of an irradiated Salmonella vaccine. Animals were immunized as described in Example 2; and colony forming units (CFU) in spleen (FIG. 1A) and liver (FIG. 1B) were determined after challenge with live S. dublin.

FIG. 2 depicts protective immunity elicited with irradiated, but not heat-killed, Listeria monocytogenes.

FIGS. 3A and 3B depicts retention of ability of lyophilized irradiated Listeria monocytogenes to induce an immune response. Animals were immunized as described in Example 3; and colony forming units (CFU) in spleen (FIG. 3A) and liver (FIG. 3B) were determined after challenge with live Listeria monocytogenes (LM).

FIG. 4 depicts activation of dendritic cells by irradiated, vs. heat-killed, Listeria monocytogenes.

FIG. 5 depicts induction of CD8⁺ T cell activation by dendritic cells activated by irradiated Listeria monocytogenes.

FIG. 6 depicts induction of CD4⁺ T cell activation by dendritic cells activated by irradiated Listeria monocytogenes.

FIG. 7 depicts the potency of selected phosphodiester ISS-ODNs.

FIG. 8 depicts predicted secondary structure of exemplary ODNs R10-53 (SEQ ID NO:13); R10-60 (SEQ ID NO:10); D-R15-8 (SEQ I NO:11); and R10-9 (SEQ ID NO:5).

FIG. 9 depicts immunostimulatory activity of ISS-ODN multimers compared with ISS-ODN monomers.

FIGS. 10A and 10B depict protein aggregation of phosphorothioate ODNs.

FIG. 11 depicts attenuation of TLR9 signaling by ISS-ODN monomers.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) reducing the incidence and/or risk of relapse of the disease during a symptom-free period; (b) relieving or reducing a symptom of the disease; (c) preventing the disease from occurring in a subject which may be predisposed to the disease but has not yet been diagnosed as having it; (d) inhibiting the disease, i.e., arresting its development (e.g., reducing the rate of disease progression); (e) reducing the frequency of episodes of the disease; and (f) relieving the disease, i.e., causing regression of the disease.

The terms “individual,” “host,” “subject,” and “patient,” used interchangeably herein, refer to a mammal, e.g., a human. Where the host is a mammal, the subject will generally be a human, but may also be a domestic livestock (e.g., horse, cattle, pigs, goats, sheep, etc.), a mammalian laboratory subject (e.g., a rodent, a lagomorph, etc.), or mammalian pet animal.

The terms, “increasing,” “inducing,” and “enhancing,” used interchangeably herein with reference to an immune response, e.g., a Th1-type immune response, refer to any increase in an immune response over background. The term includes, e.g., inducing a CTL response over an absence of a measurable CTL response; increasing a CTL response over a previously measurable CTL response.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3^{rd} ed. Amer. Pharmaceutical Assoc.

The term “bacterial biological warfare agent,” as used herein, refers generally to any bacterial agent that is developed, and/or produced, and/or used specifically for the purpose of inflicting disease and/or death upon a human population (where “human population” includes military personnel and civilian populations).

The term “bacterial biological warfare agents” further includes naturally-occurring (e.g., wild-type) bacteria as listed above; a naturally-occurring variant of any of the above-listed bacteria; and variants generated in the laboratory, including variants generated by selection, variants generated by chemical modification, and genetically modified variants (e.g., bacteria modified in a laboratory by recombinant DNA methods). Variant bacteria generated in the laboratory are referred to herein as “recombinant bacteria” or “synthetic bacteria.” Recombinant or synthetic bacterial biological warfare agents include variants of the above-listed bacteria that have increased virulence compared to a wild-type bacteria, and/or increased stability (e.g., storage stability at extreme high temperatures, and the like) compared to a corresponding wild-type bacteria, etc.

The terms “antigen” and “epitope” are well understood in the art and refer to the portion of a macromolecule which is specifically recognized by a component of the immune system, e.g., an antibody or a T-cell antigen receptor. The term “antigen” refers to a peptide, a polypeptide, a polysaccharide, a polysaccharide conjugate, a lipid, a glycolipid, a lipopolysaccharide, a glycoprotein, a lipoprotein, or other macromolecule to which an immune response can be induced in a mammalian host. As used herein, the term “antigen” encompasses antigenic epitopes, e.g., fragments of an antigen which are antigenic epitopes. Epitopes are recognized by antibodies in solution, e.g. free from other molecules. Epitopes are recognized by T-cell antigen receptor when the epitope is associated with a class I or class II major histocompatibility complex molecule.

The terms “polypeptide,” “peptide,” and “protein,” used interchangeably herein, refer to a polymeric form of amino acids of any length, which can include coded and non-coded amino acids, chemically or biochemically modified or derivatized amino acids, and polypeptides having modified peptide backbones. The term includes polypeptide chains modified or derivatized in any manner, including, but not limited to, glycosylation, formylation, cyclization, acetylation, phosphorylation, and the like. The term includes naturally-occurring peptides, synthetic peptides, and peptides comprising one or more amino acid analogs. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “tumor-associated antigen” is a term well understood in the art, and refers to surface molecules that are differentially expressed in tumor cells relative to non-cancerous cells of the same cell type. As used herein, “tumor-associated antigen” includes not only complete tumor-associated antigens, but also epitope-comprising portions (fragments) thereof. A tumor-associated antigen (TAA) may be one found in nature, or may be a synthetic version of a TAA found in nature, or may be a variant of a naturally-occurring TAA, e.g., a variant which has enhanced immunogenic properties.

An “allergen” as used herein refers to a molecule capable of provoking an immune response characterized by production of IgE. Thus, in the context of this invention, the term “allergen” refers to an antigen which triggers, in an individual who is susceptible to such (e.g., an individual who has been sensitized to the antigen), an allergic response which is mediated by IgE antibody. “Allergens” include fragments of allergens and haptens that function as allergens.

“A peptide associated with a pathogenic organism,” as used herein, is a peptide (or fragment or analog thereof) that is normally a part of a pathogenic organism, or is produced by a pathogenic organism. Generally, a peptide associated with a pathogenic organism is one that is recognized as foreign by a normal individual with a healthy, intact immune system, e.g., the peptide is displayed together with an MHC Class I molecule on the surface of a cell, where it is recognized by a CD8⁺ lymphocyte.

As used herein, the term “isolated” is meant to describe a compound of interest (e.g., an antigen, a TLR ligand, etc.) that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound (e.g., an antigen, a TLR ligand, etc.) that is removed from its natural environment and is substantially free from other components with which it is naturally associated, e.g., is at least 60% pure, at least 75% pure, at 90% pure, at least 95% pure, at least 98% pure, or at least 99% pure.

In the context of a bacterium, the term “isolated,” as used herein, refers to a bacterium that is in an environment that is different from that in which the bacterium naturally occurs. In the context of a bacterium, the term “isolated,” as used herein, is meant to include bacteria that are within samples that are substantially enriched for the bacterium of interest and/or in which the bacterium of interest is partially or substantially purified. In many embodiments, a subject irradiated bacterium is purified, e.g., is substantially free from other bacteria, and is substantially free from other components which may be undesirable (e.g., contaminants), e.g., the bacterium is at least 60% pure, at least 75% pure, at 90% pure, at least 95% pure, at least 98% pure, or at least 99% pure.

“Preventing an infectious disease,” as used herein, refers to reducing the likelihood that an individual, upon infection by a pathogenic organism, will exhibit the usual symptoms of a disease caused by a pathogenic organism.

“Treating an infectious disease,” as used herein, encompasses reducing the number of pathogenic agents in the individual (e.g., reducing viral load, reducing bacterial load) and/or reducing a parameter associated with the infectious disease, including, but not limited to, reduction of a level of a product produced by the infectious agent (e.g., a toxin, an antigen, and the like); and reducing an undesired physiological response to the infectious agent (e.g., fever, tissue edema, and the like).

The term “allergic disorder” generally refers to a disease state or syndrome whereby the body produces an immune response to environmental antigens comprising immunoglobulin E (IgE) antibodies which evoke allergic symptoms such as itching, sneezing, coughing, respiratory congestion, rhinorrhea, skin eruptions and the like. Examples of allergic diseases and disorders which can be treated by the methods of this invention include, but are not limited to, drug hypersensitivity, allergic rhinitis, ragweed pollen hayfever, urticaria, angioedema, atopic dermatitis, erythema nodosum, erythema multiforme, Stevens Johnson Syndrome, cutaneous necrotizing venulitis, bullous skin diseases, allergy to food substances and insect venom-induced allergic reactions, as well as any other allergic disease or disorder.

The terms “cancer,” “neoplasm,” and “tumor,” are used interchangeably herein to refer to cells which exhibit relatively autonomous growth, so that they exhibit an aberrant growth phenotype characterized by a significant loss of control of cell proliferation. Cancerous cells can be benign or malignant.

The terms “CD4⁺-deficient” and “CD4⁺-low” are used interchangeably herein, and, as used herein, refer to a state of an individual in whom the number of CD4⁺ T lymphocytes is reduced compared to an individual with a healthy, intact immune system. CD4⁺ deficiency includes a state in which the number of functional CD4⁺ T lymphocytes is less than about 600 CD4⁺ T cells/mm³ blood; a state in which the number of functional CD4⁺ T cells is reduced compared to a healthy, normal state for a given individual; and a state in which functional CD4⁺ T cells are completely absent.

As used herein, a “CD4⁺-deficient individual” is one who has a reduced number of functional CD4⁺-T cells, regardless of the reason, when compared to an individual having a normal, intact immune system. In general, the number of functional CD4⁺-T cells that is within a normal range is known for various mammalian species. In human blood, e.g., the number of functional CD4⁺-T cells which is considered to be in a normal range is from about 600 to about 1500 CD4⁺-T cells/mm³ blood. An individual having a number of CD4⁺-T cells below the normal range, e.g., below about 600/mm³, may be considered “CD4⁺-deficient.” Thus, a CD4⁺-deficient individual may have a low CD4⁺ T cell count, or even no detectable CD4⁺ T cells. A CD4⁺-deficient individual includes an individual who has a lower than normal number of functional CD4⁺-T cells due to a primary or an acquired immunodeficiency.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a bacterium” includes a plurality of such bacteria and reference to “the formulation” includes reference to one or more formulations and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides a bacterial composition comprising lethally irradiated bacteria. A subject bacterial composition is useful for inducing an immune response to 1) a live pathogen; 2) an exogenous antigen synthesized by a genetically modified live bacteria; 3) a co-administered antigen; or 4) a co-administered inactivated microbial agent such as a dead virus. Thus, the present invention provides immunogenic compositions comprising lethally irradiated bacteria. In many embodiments, a subject bacterial composition is formulated for mucosal delivery. The present invention further provides methods of preparing a subject immunogenic composition. The present invention further provides a method of inducing an immune response to a microbial pathogen or an antigen in an individual, the method generally involving administering a subject immunogenic composition to a mucosal tissue of the individual.

The present invention is based in part on the observation that vaccination with lethally irradiated bacteria, in contrast to heat/formalin inactivation, protects animals from subsequent lethal challenge as effectively as vaccination with low inoculum of viable bacteria. The use of lethally irradiated bacteria as a vaccine platform has several advantages over vaccination with purified antigens administered via injection and over vaccination with live, attenuated bacteria. These advantages, as discussed in more detail below, include: 1) ease of development and preparation; 2) stimulation of a multi-faceted immune response; 3) accessibility to target population; 4) mucosal routes of administration that avoid the need for using needles; 5) increased patient compliance; 6) storage stability; and 7) safety.

Using lethally irradiated bacteria for vaccination purposes provides a simple technical breakthrough that removes critical barriers in vaccine development, e.g., the discovery of conserved protective antigens and their formulation for vaccination. Use of lethally irradiated bacteria as vaccine platforms obviates the need for antigen discovery, antigen re-design as well as antigen purification. The manufacturing of each individual vaccine according to the present invention is inexpensive and can be easily and rapidly produced in high quantities.

Use of lethally irradiated bacteria provides sufficient amounts of unmodified antigens in their natural conformation. After being processed by antigen-presenting cells, lethally irradiated bacteria provide multiple epitopes for the induction of neutralizing secretory IgA (sIgA) and IgG antibodies, as well as CD4 and CD8 cellular immune responses. The vaccines themselves are equipped with natural adjuvants (e.g., toll-like receptor, or TLR, agonists) that induce maturation of dendritic cells (DC); and facilitate antigen uptake, processing, and antigen presentation.

Subject immunogenic compositions provide a simplified and shortened vaccination protocol that is necessary for a population with limited accessibility and/or access to health care facilities. The use of heat stable, lethally irradiated bacteria significantly reduce the logistical costs required for a successful vaccination program. These attributes are mandatory for successful vaccination programs especially in developing countries.

Lethally irradiated bacteria of the instant invention are in many embodiments applied to mucosal surfaces. Thus, routes of administration include oral, nasal, and inhalational routes of administration. Such routes of administration are less expensive than injection (e.g., subcutaneous injection; intramuscular injection), which typically is carried out using a needle and syringe system. Furthermore, by avoiding the use of needle and syringe injection systems, one avoids the possibility of needle re-use, and the risks associated with such re-use (e.g., infection with a pathogenic virus such as hepatitis B virus, hepatitis C virus, human immunodeficiency virus, etc.).

Mucosal administration increases compliance in the target population. For example, individuals who may have an aversion to being injected using a needle are more likely to comply with a vaccination program that uses oral, nasal, or inhalational administration. Furthermore, the use of lethally-irradiated bacteria allows incorporation of such bacteria into a palatable carrier (e.g., a flavored carrier such as candy; a palatable beverage, etc.), further increasing target population compliance, especially in pediatric target populations. In addition, because the subject bacterial compositions need not be administered by a medical professional, the subject compositions are well suited for administration to individuals who may be averse to traveling, or who may be unable to travel long distances to reach a medical professional who, in some areas, may be far away and relatively inaccessible to the target population. In such instances, because a bacterial composition can be self administered (e.g., orally, intranasally, etc.), target population compliance is increased.

Because subject bacterial compositions are lethally irradiated, and in some cases lyophilized, they are storage stable. Storage stability reduces costs associated with transportation and storage. This feature makes the subject immunogenic compositions ideal for developing countries. This features also allows stockpiling of vaccines, e.g., in preparation for an unexpected surge in the need for such a composition, e.g., a bioterror attack.

Unlike bacterial vaccines that have been proposed that involve use of live, attenuated bacteria, the subject bacterial compositions comprise dead bacteria. This feature makes them safe for use in humans, and particularly for certain populations, e.g., infants, children, and individuals who are immunocompromised, e.g., that have less than the normal range of CD4⁺ T lymphocytes.

Bacterial Compositions

The present invention provides bacterial compositions comprising lethally-irradiated bacteria. Subject bacterial compositions induce an immune response in a mammalian host to an antigen. Thus, the present invention provides immunogenic compositions comprising a subject lethally-irradiated bacteria. In some embodiments, the irradiated bacteria induce an immune response to an endogenous antigen, e.g., one that is normally synthesized by the bacteria. In these embodiments, the irradiated bacteria is generated using live, pathogenic bacteria. In other embodiments, subject irradiated bacteria induce an immune response to an exogenous antigen, e.g., one that is not normally synthesized by the bacteria. In some of these embodiments, the exogenous antigen is provided in the composition in admixture with lethally-irradiated bacteria. In other embodiments where the antigen is an exogenous antigen, the antigen is synthesized by bacteria that are genetically modified to include a polynucleotide that encodes the exogenous antigen.

A subject bacterial composition comprises irradiated bacteria that are unable to replicate. In some embodiments, irradiated bacteria are produced by first lyophilizing live bacteria; then lethally irradiating the lyophilized bacteria. In some embodiments, irradiated bacteria are produced by first lethally irradiating live bacteria; then lyophilizing the lethally irradiated bacteria. The term “irradiated bacteria,” as used herein, refers to bacteria that are initially live, but are lethally irradiated, and in some embodiments are lyophilized, then lethally irradiated, or lethally irradiated, then lyophilized. The process of preparing a subject bacterial composition results in bacteria that are dead, e.g., are unable to grow and divide. In some embodiments, lethally irradiated bacteria are metabolically inactive (e.g., the lethally irradiated bacteria do not synthesize any macromolecules or other compounds).

A subject bacterial composition is capable, when administered to a mucosal tissue of a mammalian subject, of stimulating an immune response in the subject to an antigen. Where a subject bacterial composition comprises lethally-irradiated bacteria that are made by lethally irradiating (or lyophilizing, then lethally irradiating; or lethally irradiating, then lyophilizing) live, pathogenic bacteria, a subject bacterial composition induces an immune response to live pathogenic bacteria of the same and/or related species and of the same and/or related strains. Where a:subject bacterial composition comprises lethally-irradiated bacteria and an exogenous antigen(s), a subject bacterial composition induces an immune response to the exogenous antigen(s). Where the exogenous antigen is an antigen of a microbial pathogen (e.g., a bacterial pathogen, a viral pathogen, a parasite (such as a helminth, a protozoa, etc.), a mycobacterial pathogen, etc.), a subject bacterial composition induces an immune response to the microbial pathogen. Where the exogenous antigen is a tumor-associated antigen, a subject bacterial composition induces an immune response to a tumor cell bearing the tumor-associated antigen. Where the exogenous antigen is an allergen, a subject bacterial composition reduces a Th2-type immune response, reducing production of IgE antibodies in response to exposure to the allergen. Use of a subject bacterial composition in the treatment of allergic disorders is discussed in more detail below.

Where a subject bacterial composition comprises lethally-irradiated bacteria that are made by irradiating (or lyophilizing, then irradiating) live, pathogenic bacteria, the term “stimulating an immune response,” as used herein, includes one or more of the following: 1) stimulating production of antibodies that bind specifically to the lethally-irradiated bacteria as well as a live bacterium of the same species and/or related species and/or strains; 2) stimulating a CD4 T cell response specific for the irradiated bacterium as well as for a live bacterium of the same species and/or related species and/or related strains; 3) stimulating a CD8 cytotoxic T lymphocyte (CTL) immune response specific for the irradiated bacteria as well as for live bacteria of the same species and/or related species and/or related strain; 4) stimulating a protective immune response following challenge with live bacteria of the same species and/or related species and/or related strain as the irradiated bacteria; 5) stimulation production of antibodies that bind specifically an endogenous antigen produced by the lethally-irradiated bacteria before they are lethally irradiated, and/or that cross-react with an antigen produced by a live bacterium of the same species and/or related species and/or strains; and 6) stimulating a CD4 T cell response specific for an endogenous antigen produced by the lethally-irradiated bacteria before they are lethally irradiated, and/or that cross-react with an antigen produced by a live bacterium of the same species and/or related species and/or strains.

Where a subject bacterial composition comprises lethally-irradiated bacteria and an exogenous antigen(s), the term “stimulating an immune response,” as used herein, includes one or more of the following: 1) stimulating production of antibodies that bind specifically to the exogenous antigen(s); 2) stimulating a CD4 cell response specific for the exogenous antigen; 3) stimulating a CD8 cell response specific for the exogenous antigen; 4) stimulating a protective immune response following challenge with a live microbial pathogen that produces the exogenous antigen. Stimulation of an immune response by a subject immunogenic composition is discussed in more detail below.

In some embodiments, effective amounts of a subject bacterial composition are amounts that are effective to increase an antigen-specific CTL response by at least about 10%, at least about 20%, at least about 25%, at least about 50%, at least about 75%, at least about 100% (or two-fold), at least about 5-fold, at least about 10-fold, at least about 20-fold, at least about 50-fold, or at least about 100-fold or more, when compared to a suitable control. In an experimental animal system, a suitable control may be a genetically identical animal not treated with the subject composition. In non-experimental systems, a suitable control may be the level of antigen-specific CTL present before administering the subject composition. Other suitable controls may be a placebo control.

In some embodiments, effective amounts of a subject bacterial composition are amounts that are effective to increase an antigen-specific antibody response (other than IgE) by at least about 10%, at least about 20%, at least about 25%, at least about 50%, at least about 75%, at least about 100% (or two-fold), at least about 5-fold, at least about 10-fold, at least about 20-fold, at least about 50-fold, or at least about 100-fold or more, when compared to a suitable control. In an experimental animal system, a suitable control may be a genetically identical animal not treated with the subject composition. In non-experimental systems, a suitable control may be the level of antigen-specific antibody present before administering the subject composition. Other suitable controls may be a placebo control.

In some embodiments, effective amounts of a subject bacterial composition are amounts that are effective to increase an antigen-specific CD4 response by at least about 10%, at least about 20%, at least about 25%, at least about 50%, at least about 75%, at least about 100% (or two-fold), at least about 5-fold, at least about 1 0-fold, at least about 20-fold, at least about 50-fold, or at least about 100-fold or more, when compared to a suitable control. In an experimental animal system, a suitable control may be a genetically identical animal not treated with the subject composition. In non-experimental systems, a suitable control may be the level of antigen-specific CD4 present before administering the subject composition. Other suitable controls may be a placebo control.

Whether an antibody response to an antigen has been induced in an individual is readily determined using standard assays. For example, immunological assays such as enzyme-linked immunosorbent assays (ELISA), radioimmunoassay (RIA), immunoprecipitation assays, and protein blot (“Western” blot) assays; and neutralization assays (e.g., neutralization of viral infectivity in an in vitro or in vivo assay); can be used to detect the presence of antibody specific for a microbial antigen in a bodily fluid or other biological sample, e.g., the serum, secretion, or other fluid, of an individual.

Whether a CD4 immune response to an antigen has been induced in an individual is readily determined using standard assays, e.g., fluorescence-activated cell sorting (FACS) (see, e.g., Waldrop et al. (1997) J. Clin. Invest. 99:1739-1750); intracellular cytokine assays that detect production of cytokines following antigen stimulation (see, e.g., Suni et al. (1998) J. Immunol. Methods 212:89-98; Nomura et al. (2000) Cytometry 40:60-68; Ghanekar et al. (2001) Clin. Diagnostic Lab. Immunol. 8:628-631); MHC-peptide multimer staining assays, e.g., use of detectably labeled (e.g., fluorescently labeled) soluble MHC Class II/peptide multimers (see, e.g., Bill and Kotzin (2002) Arthritis Res. 4:261-265; Altman et al. (1996) Science 274:94-96; and Murali-Krishna et al. (1998) Immunity 8:177-187); enzyme-linked immunospot (ELISPOT) assays (see, e.g., Hutchings et al. (1989) J. Immunol. Methods 120:1-8; and Czerkinsky et al. (1983) J. Immunol Methods 65:109-121); and the like. As one non-limiting example of an intracellular cytokine assay, whole blood is stimulated with antigen and co-stimulating antibodies (e.g., anti-CD28, anti-CD49d) for 2 hours or more; Brefeldin A is added to inhibit cytokine secretion; and the cells are processed for FACS analysis, using fluorescently labeled antibodies to CD4 and to cytokines such as TNF-α, IFN-γ and IL-2.

Whether an antigen-specific CD8 (e.g., cytotoxic T cell; “CTL”) response is induced to an intracellular pathogen can be determined using any of a number of assays known in the art, including, but not limited to, measuring specific lysis by CTL of target cells expressing an antigen of the intracellular pathogen on their surface, which target cells have incorporated a detectable label which is released from target cells upon lysis, and can be measured, using, e.g., an assay such as that described in the Examples, a ⁵¹Cr-release assay; a lanthanide fluorescence-based cytolysis assay; and the like.

Lethally-Irradiated Bacteria that Induce an Immune Response to an Endogenous Antigen

The present invention provides compositions comprising lethally-irradiated bacteria (e.g., lyophilized, then lethally-irradiated bacteria; or lethally-irradiated, then lyophilized), that induce an immune response to an endogenous antigen, e.g., an antigen that is synthesized by live bacteria of the same strain in nature. Typically, the lethally-irradiated bacteria are generated using a live, pathogenic bacteria. Live, pathogenic bacteria are lethally irradiated, to generate a subject bacterial composition. In some embodiments, live, pathogenic bacteria are lyophilized then lethally irradiated, to generate a subject bacterial composition. The compositions are useful for stimulating an immune response in an individual to a live, pathogenic bacterium.

In some embodiments, a subject composition comprises lethally-irradiated irradiated bacteria, formulated without any additional adjuvant. In other embodiments, a subject composition comprises lethally irradiated bacteria formulated in admixture with at least one monomeric toll-like receptor (TLR) ligand. In other embodiments, a subject composition comprises lethally irradiated bacteria formulated in admixture with at least one multimeric TLR ligand. In other embodiments, a subject composition comprises lethally irradiated bacteria formulated in admixture with at least one chimeric TLR ligand. In still other embodiments, a subject composition comprises lethally irradiated bacteria that is conjugated to at least one TLR ligand.

In some embodiments, a subject bacterial composition comprises:
a) lethally irradiated, lyophilized bacteria that are prepared by a process of either i) lethally irradiating pathogenic bacteria; and ii) lyophilizing the lethally-irradiated bacteria; or i) lyophilizing pathogenic bacteria; and ii) lethally irradiating the lyophilized bacteria; and
b) a pharmaceutically acceptable excipient. In some of these embodiments, the composition further comprises an adjuvant.

In some embodiments, a subject bacterial composition comprises:
a) lethally irradiated, lyophilized bacteria that are prepared by a process of either i) lethally irradiating pathogenic bacteria; and ii) lyophilizing the lethally-irradiated bacteria; or i) lyophilizing pathogenic bacteria; and ii) lethally irradiating the lyophilized bacteria; and
b) a monomeric TLR ligand (e.g., a TLR agonist). In many of these embodiments, the TLR ligand is a synthetic TLR ligand that is purified. In many embodiments, the lethally-irradiated, lyophilized bacteria are in admixture with the TLR ligand. In many embodiments, the TLR ligand is a TLR9 ligand. In some of these embodiments, the composition further comprises an adjuvant.

In some embodiments, a subject bacterial composition comprises:
a) lethally irradiated, lyophilized bacteria that are prepared by a process of either i) lethally irradiating pathogenic bacteria; and ii) lyophilizing the lethally-irradiated bacteria; or i) lyophilizing pathogenic bacteria; and ii) lethally irradiating the lyophilized bacteria; and
b) a monomeric TLR ligand, where the lethally-irradiated, lyophilized bacteria are in admixture with the monomeric TLR ligand, and the TLR ligand is a TLR9 ligand, e.g., a nucleic acid comprising 5′CG 3′. In many of these embodiments, the monomeric TLR9 ligand is a synthetic TLR9 ligand that is purified. In some of these embodiments, the composition further comprises an adjuvant.

In some embodiments, a subject bacterial composition comprises:
a) lethally irradiated, lyophilized bacteria that are prepared by a process of either i) lethally irradiating pathogenic bacteria; and ii) lyophilizing the lethally-irradiated bacteria; or i) lyophilizing pathogenic bacteria; and ii) lethally irradiating the lyophilized bacteria; and
b) a multimeric TLR ligand, where the lethally-irradiated, lyophilized bacteria are in admixture with the multimeric TLR ligand, and the TLR ligand is a TLR9 ligand, e.g., a nucleic acid comprising 5′CG 3′. In many of these embodiments, the multimeric TLR9 ligand is a synthetic TLR9 ligand that is purified. In some of these embodiments, the composition further comprises an adjuvant.

In some embodiments, a subject bacterial composition comprises:
a) lethally irradiated, lyophilized bacteria that are prepared by a process of either i) lethally irradiating pathogenic bacteria; and ii) lyophilizing the lethally-irradiated bacteria; or i) lyophilizing pathogenic bacteria; and ii) lethally irradiating the lyophilized bacteria; and
b) a chimeric TLR ligand. In many of these embodiments, the chimeric TLR ligand is a synthetic TLR ligand that is purified. In some of these embodiments, the composition further comprises an adjuvant.
   Bacteria

Bacteria that are suitable for including in a subject bacterial composition are any aerobic or anaerobic bacteria that are pathogenic or that can be rendered pathogenic (e.g., by manipulation in the laboratory, e.g., by selection, genetic engineering, etc.). Of particular interest in many embodiments are bacteria that are pathogenic in humans. Of particular interest in some embodiments are bacteria that are etiologic agents of enteric diseases. Of particular interest in some embodiments are bacteria that are pathogenic in non-human animals, e.g., bacteria that are pathogenic in livestock (e.g., sheep, cattle, goats, pigs, etc.); bacteria that are pathogenic in race horses; and the like.

Suitable gram positive bacteria include, but are not limited to pathogenic Pasteurella species, Staphylococci species, and Streptococcus species, Pneumococcus sp., Listeria sp. Also suitable for use in a subject bacterial composition is any gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Clostridia, Klebsiella, Francisella, Anthrax, Mycobacterium sp., Mycoplasma sp, Rickettsia sp., Spirochetal sp., and Bacterioides. See, e.g., Schaechter, M, H. Medoff, D. Schlesinger, Mechanisms of Microbial Disease. Williams and Wilkins, Baltimore (1989).

Specific examples of infectious, pathogenic bacteria include but are not limited to: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of suitable pathogenic E. coli strains are: ATCC No. 31618, 23505, 43886, 43892, 35401, 43896, 33985, 31619 and 31617. Non-limiting examples of mycobacteria include Mycobacterium tuberculosis, M. avium (or M. avium-intracellulare), M. leprae (particularly M. leprae infection leading to tuberculoid leprosy), M. kansasii, M. fortuitum, M. chelonae, and M. abscessus.

In some embodiments, a subject bacterial composition comprises lethally irradiated bacteria generated by irradiating a bacterial bioterror agent. Examples of possible bacterial bioterror agents listed by the U.S. Centers for Disease Control and Prevention (CDC) include Bacillus anthracis, Brucella sp., Vibrio cholerae, Coxiella burnetti, E. coli O157:H7, Clostridium perfringens, Salmonella sp., Shigella sp., Francisella tularensis, Yersinia pestis, Burkholderia mallei, Burkholderia pseudomallei, Chlamydophila psittaci, Rickettsia prowazekii, and the like.

In some embodiments, a subject bacterial composition comprises a mixture of lethally irradiated bacteria of two or more different species, or two or more different strains or serotypes of the same species. In some embodiments, a subject bacterial composition comprises a mixture of two or more different bacterial species or two or more different strains of the same species, of bacteria that are the etiologic agents of diarrhea. In some embodiments, a subject bacterial composition comprises a mixture of two or more different bacterial species or two or more different strains of the same species, of bacteria that are the etiologic agents of pneumonia.

Compositions Comprising Lethally Irradiated Bacteria and an Exogenous Antigen

The present invention provides compositions comprising lethally irradiated bacteria (e.g., lyophilized, lethally irradiated bacteria) and an antigen, e.g., an exogenous antigen not normally synthesized by a live bacterium of the same strain in nature. In some embodiments, a subject composition comprises lethally irradiated bacteria and an antigen in admixture. In other embodiments, a subject composition comprises lethally irradiated bacteria and an antigen produced by the bacteria, where the bacteria, when live, was genetically modified with a polynucleotide that comprises a nucleotide sequence that encodes the antigen, and where the bacteria, when live, was cultured under conditions that favor production of the antigen. Thus, e.g., in some embodiments, a subject bacterial composition is prepared by either i) lyophilizing a bacterium that has been genetically modified to produced an exogenous antigen, and that has been cultured under conditions that provide for production of the exogenous antigen; and ii) lethally irradiating the lyophilized bacterium; or i) lethally irradiating a bacterium that has been genetically modified to produced an exogenous antigen and that has been cultured under conditions that provide for production of the exogenous antigen; and ii) lyophilizing the lethally irradiating bacterium.

Irradiated Bacteria Admixed with Antigen

In some embodiments, a subject composition comprises lethally irradiated bacteria and an antigen in admixture. The lethally irradiated bacteria serve as an adjuvant. When administered to a mammalian subject, a subject composition comprising irradiated bacteria and an antigen induces an immune response to the antigen in the mammalian subject.

Antigens

Where a subject composition comprises lethally irradiated bacteria and an antigen in admixture, suitable antigens include, but are not limited to, allergens, microbial antigens (e.g., viral antigens, bacterial antigens, fungal antigens, protozoan antigens, helminth antigens, yeast antigens, etc.), tumor antigens, and the like.

In some embodiments, the antigen is purified, or partially purified. In other embodiments, the antigen is provided as a crude extract. Antigens may be synthesized chemically or enzymatically, may be produced recombinantly, may be isolated from a natural source, or a combination of the foregoing. In some embodiments, the antigen is a whole microbial pathogen that has been inactivated. For example, a “viral antigen” includes a dead virus.

In some embodiments, a subject composition comprises lethally irradiated bacteria and at least two different antigens. In a particular embodiment, a subject composition comprises antigens from two, three, four, five, or more, different Streptococcal strains.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Well-established recombinant DNA techniques can be employed for production of polypeptides, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

In many embodiments, the antigen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

Microbial Antigens

Suitable viral antigens include those associated with (e.g., synthesized by) viruses of one or more of the following groups: Retroviridae (e.g. human immunodeficiency viruses, such as HIV-1 (also referred to as HTLV-III, LAV or HTLV-III/LAV, or HIV-III; and other isolates, such as HIV-LP; Picomaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses.

As mentioned above, in some embodiments, the viral antigen is an isolated viral antigen. In other embodiments, the viral antigen is a whole, inactivated virus. Methods of inactivating a whole virus are well known in the art; any known method can be used to inactivate a virus. Methods of inactivating a virus include use of photoreactive compounds; oxidizing agents; irradiation (e.g., UV irradiation; γ-irradiation); combinations of riboflavin and UV irradiation; solvent-detergent treatment (e.g., treatment with organic solvent tri-N-butyl-phosphate with a detergent such as Tween 80); polyethylene glycol treatment; pasteurization (heat treatment); and low pH treatment; mild enzymatic treatment with pepsin or trypsin; Methylene blue (MB) phototreatment; treatment with Dimethylmethylene blue (DMMB) and visible light; treatment with S-59, a psoralen derivative and UVA illumination; and the like.

Suitable bacterial antigens include antigens associated with (e.g., synthesized by and endogenous to) any of a variety of pathogenic bacteria, including, e.g., pathogenic gram positive bacteria such as pathogenic Pasteurella species, Staphylococci species, and Streptococcus species; and gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Francisella and Bacterioides. See, e.g., Schaechter, M, H. Medoff, D. Schlesinger, Mechanisms of Microbial Disease.

Williams and Wilkins, Baltimore (1989)).

Suitable antigens associated with (e.g., synthesized by and endogenous to) infectious pathogenic fungi include antigens associated with infectious fungi including but not limited to: Cryptococcus neoformans, Histoplasma capsulatum, Coccidioides immitis, Blastomyces dermatitidis, and Candida albicans, Candida glabrata, Aspergillus fumigata, Aspergillus flavus, and Sporothrix schenckii.

Suitable antigens associated with (e.g., synthesized by and endogenous to) pathogenic protozoa, helminths, and other eukaryotic microbial pathogens include antigens associated with protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Suitable antigens include antigens associated with (e.g., synthesized by and endogenous to) pathogenic microorganisms such as: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of pathogenic E. coli strains are: ATCC No. 31618, 23505, 43886, 43892, 35401, 43896, 33985, 31619 and 31617.

Any of a variety of polypeptides or other antigens associated with intracellular pathogens may be included in a subject composition. Polypeptides and peptide epitopes associated with intracellular pathogens are any polypeptide associated with (e.g., encoded by) an intracellular pathogen, fragments of which are displayed together with MHC Class I molecule on the surface of the infected cell such that they are recognized by, e.g., bound by a T-cell antigen receptor on the surface of, a CD8⁺ lymphocyte. Polypeptides and peptide epitopes associated with intracellular pathogens are known in the art and include, but are not limited to, antigens associated with human immunodeficiency virus, e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP-1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; Plasmodium antigens (e.g., gp190/MSP1, and the like); etc.

Tumor-Associated Antigens

Any of a variety of known tumor-specific antigens or tumor-associated antigens (TAA) can be included in a subject composition. The entire TAA may be, but need not be, used. Instead, a portion of a TAA, e.g., an epitope, may be used. Tumor-associated antigens (or epitope-containing fragments thereof) which may be used into YFV include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, and p21ras. A synthetic analog of any TAA (or epitope thereof), including any of the foregoing, may be used. Furthermore, combinations of one or more TAAs (or epitopes thereof) may be included in the composition.

Allergens

Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); Other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); cccupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Altemaria (Altemaria altemata); Alder; Alnus (Alnus gultinoas); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherun elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Bacteria

Where a subject bacterial composition comprises irradiated bacteria and an antigen, (including, e.g., any killed microbial agent such as a dead virus), in some embodiments irradiated bacteria are generated by irradiating (or lyophilizing, then irradiating; or irradiating, then lyophilizing) live, pathogenic bacteria. Suitable live, pathogenic bacteria are those discussed above.

Where a subject composition comprises irradiated bacteria and an antigen, in some embodiments irradiated bacteria are generated by irradiating (or lyophilizing, then irradiating; or irradiating, then lyophilizing) live, probiotic bacteria. Suitable probiotic bacteria include, but are not limited to, bacteria of various species, including lactobacillus species, e.g., Lactobacillus acidophilus, L. plantarum, L. casei, L. rhamnosus, L. delbrueckii (including subspecies bulgaricus), L. reuteri, L. fermentum, L. brevis, L. lactis, L. cellobiosus, L. GG, L. gasseri, L. johnsonii, and L. plantarum; bifidobacterium species, e.g., Bifidobacterium bifidum, B. infantis, B. longum, B. thermophilum, B. adolescentis, B. breve, B. animalis; streptococcus species, e.g., Streptococcus lactis, S. cremoris, S. salivarius (including subspecies thermophilus), and S. intermedius; Leuconostoc species; Pediococcus species; Propionibacterium species; Bacillus species; non-enteropathogenic Escherichia species, e.g., non-enteropathogenic Escherichia coli, e.g., E. coli Nissle, and the like; and Enterococcus species such as Enterococcus faecalis, and E. faecium. Other suitable probiotic bacteria are known in the art, and have been described. See, e.g., U.S. Pat. No. 5,922,375.

Genetically Modified, Irradiated Bacteria

In some embodiments, a subject composition comprises irradiated bacteria and an antigen produced by the bacteria, where the bacteria, when live, were genetically modified with a polynucleotide that comprises a nucleotide sequence that encodes the antigen, and where the bacteria, when live, were cultured under conditions that provide for production of the antigen.

A wide variety of nonvirulent, non-pathogenic bacteria may be used; e.g., relatively well characterized bacterial strains, particularly nonvirulent, non-pathogenic strains of various bacteria, including, but not limited to, E. coli, such as MC4100, MC1061, DH5α; Listeria monocytogenes, Shigella (e.g., Shigella flexneri, S. dysenteriae, S. sonnei, S. boydii), Haemophilus influenzae, mycobacterium (e.g., Mycobacterium tuberculosis), Yersinia enterocolitica, Klebsiella pneumoniae, Pasteurella multocida, Salmonella (e.g., S. typhi, S. typhimurium), Bacillus subtilis, etc. See, e.g., U.S. Pat. Nos. 6,599,502, and 6,537,558. Attenuated Shigella as a delivery vehicle has been described in the literature. See, e.g., Sizemore et al. (1995) Science 270:299-302. Attenuated E. coli as a delivery vehicle has been described in the literature. See, e.g., Schweder et al. (1995) Appl. Microbiol. Biotechnol. 42:718-723. Attenuated Salmonella as a delivery vehicle has been described in the literature. See, e.g., U.S. Pat. No. 6,585,975.

Specific examples of Salmonella vectors include S. typhi mutant strains, for example, those discussed in U.S. Pat. No. 6,585,975, e.g., CVD908 S. typhi Ty2 ΔaroC/ΔaroD (Hone et al., Vaccine 9:810-816, 1991), CVD908-htrA S. typhi Ty2 ΔaroC/ΔaroD/ΔhtrA; BRD1116 S. typhi Ty2 ΔaroA/ΔaroC/ΔhtrA; S. typhi ΔaroA/ΔaroE; S. typhi Ty2 ΔaroA/ΔaroC Km-R; and S. typhi ΔaroA/ΔaroD.

Specific examples of S. typhimurium mutant strains that can be used in the invention include, for example, those discussed in U.S. Pat. No. 6,585,975, e.g., BRD509 S. typhimurium ΔaroA/ΔaroD; BRD807 S. typhimurium ΔaroA/ΔhtrA; BRD698; and BRD726.

Typically, an attenuated bacterial delivery vehicle is genetically modified by introducing into the bacterium a polynucleotide expression vector comprising a nucleotide sequence that encodes a heterologous polypeptide (e.g., an exogenous polypeptide, one that is not produced by the bacterium in nature). Expression vectors are introduced into bacteria using standard methods, e.g., calcium phosphate precipitation, electroporation, and the like.

Genetically modified bacteria are then cultured under conditions and for a suitable period of time that allow for production of the exogenous antigen. Such conditions are well known in the art. After the exogenous antigen has been synthesized, the bacteria are irradiated (or lyophilized, then irradiated), as discussed below.

Lyophilization and Irradiation

A subject bacterial composition comprises bacteria that are lethally irradiated. Bacteria are irradiated at an energy and for a period of time sufficient to render the bacteria non-viable, e.g., such that growth in in vitro culture is undetectable using standard methods. In some embodiments, bacteria are lyophilized, then lethally irradiated. In other embodiments, bacteria are irradiated, then lyophilized. The lethally-irradiated bacteria in a subject composition are dead. In some embodiments, the starting material are live, pathogenic bacteria. In other embodiments, the starting material are live, probiotic bacteria. In other embodiments, the starting material are live, recombinant (genetically modified) bacteria.

Thus, the present invention provides methods of preparing a subject bacterial composition. In some embodiments, the methods involve lyophilizing a population of live bacteria, generating a lyophilisate; then irradiating the lyophilized bacteria (the lyophilisate). The bacteria in the lyophilisate are irradiated for a period of time and at an energy sufficient to kill the bacteria. In other embodiments, the methods involve lethally irradiating a population of live bacteria; then lyophilizing the lethally irradiated bacteria. In particular, a population of bacteria (live bacteria; or lyophilized bacteria) is irradiated for a period of time and at an energy sufficient to kill at least 99%, at least 99.9%, at. least 99.99%, at least 99.999%, or 100% of the bacteria in a population of bacteria.

Typically, isolated bacteria are grown in in vitro culture in a suitable medium and at a suitable temperature, to a desired density. Once a sufficient number of bacteria are obtained, the bacteria are irradiated, lyophilized then irradiated, or irradiated then lyophilized.

Lyophilization is carried out using standard methods. See, e.g., T. A. Jennings (1999) “Lyophilization: Introduction and Basic Principles” Interpharm Press. In some embodiments, bacteria are lyophilized in a medium comprising one or more cryoprotectants such as peptone, adonitol, sodium glutamate, glycerol, lactose, gelatin, trehalose, sucrose, glucose, and dextran.

Bacteria (either live bacteria in culture; or lyophilized bacteria) are then lethally irradiated. In some embodiments, the irradiation is ionizing radiation. Gamma radiation is an example of ionizing radiation. ¹³⁷CS is a suitable source of gamma irradiation. For example, the bacteria are irradiated using gamma irradiation in an amount of from about 5 kiloGray (kGy) to about 50 kGy, from about 10 kGy to about 20 kGy, from about 20 kGy to about 40 kGy, or from about 25 kGy to about 35 kGy.

Bacteria are irradiated for a period of time of from about 15 seconds to about 96 hours, e.g., from about 15 seconds to about 1 minute, from about 1 minute to about 15 minutes, from about 15 minutes to about 30 minutes, from about 60 minutes to about 90 minutes, from about 90 minutes to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, from about 36 hours to about 48 hours, from about 48 hours to about 60 hours, from about 60 hours to about 72 hours, from about 72 hours to about 84 hours, or from about 84 hours to about 96 hours. The total amount of irradiation and the duration of irradiation can be adjusted, depending on various factors, e.g., the number of bacteria being irradiated. The total amount of irradiation and the duration of irradiation that results in bacteria that are non-viable (e.g., are unable to grow in in vitro culture) are readily determined by those of ordinary skill in the art.

In other embodiments, the radiation is ultraviolet (UV) radiation. For example, the probiotic bacteria are exposed to UV radiation of from about 2000 μW sec/cm² to about 1,000 μW sec/cm².

Viability of the bacteria is reduced by at least about 95%, or at least about 99%, or more, such that fewer than about 5%, or fewer than about 1%, or fewer, of the bacteria in the formulation are viable. In some embodiments, 100% of the bacteria are dead, e.g., are unable to grow in in vitro culture.

Viability of bacteria is determined using any known method. For example, bacteria are contacted with a membrane-permeant fluorescent dye (e.g., SYTO 9, SYTOX, and the like) that labels live bacteria with green fluorescence; and membrane-impermeant propidium iodide that labels membrane-compromised bacteria with red fluorescence. Roth et al. (1997) Appl. Environ. Microbiol. 63:2421-2431; Lebaron et al. (1998) Appl. Environ. Microbiol. 64:2697-2700; and Braga et al. (2003) Antimicrob. Agents Chemother. 47:408-412. Bacterial viability is also determined by plating the bacteria on an agar plate containing requisite nutritional supplements, and counting the number of colonies formed (colony forming units, cfu).

Subject bacterial compositions are stable at temperatures from about 4° C. to about 80° C., e.g., from about 4° C. to about 10° C., from about 10° C. to about 15°, from about 15° C. to about 20° C., from about 20° C. to about 30° C., from about 30° C. to about 40° C.. from about 40° C to about 50° C., from about 50° C. to about 60° C., from about 60° C. to about 70° C., from about 70° C. to about 80° C., from about 20° C. to about 70° C., from about 25° C. to about 65° C., from about 30° C. to about 60° C., or from about 35° C. to about 55° C. for a period of time of from about 1 week to about 10 years or longer, e.g., from about 1 week to about 2 weeks, from about 2 weeks to about 4 weeks, from about 1 month to about 2 months, from about 2 months to about 4 months, from about 4 months to about 6 months, from about 6 months to about 8 months, from about 8 months to about 12 months, from about 1 year to about 2 years, from about 2 years to about 5 years, from about 5 years to about 7 years, or from about 7 years to about 10 years, or longer.

Formulations, Dosages, Routes of Administration

The present invention provides compositions, including pharmaceutical compositions (e.g., immunogenic compositions) comprising a subject bacterial composition. Subject formulations, dosages, and routes of administration are described below.

Formulations

A subject composition may include a buffer, which is selected according to the desired use of the subject composition, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995) Mack Publishing Co.; A. Gennaro (2000) “Remington: The Science and Practice of Pharmacy”, 20^{th} edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

Pharmaceutical compositions can be prepared in various forms, such as granules, tablets, lozenges, pills, suppositories, capsules, suspensions, sprays, suppositories, transdermal applications (e.g., patches, etc.), salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

When used as an immunogenic composition, a subject bacterial composition can be formulated in a variety of ways. In general, an immunogenic composition of the invention is formulated according to methods well known in the art using suitable pharmaceutical carrier(s) and/or vehicle(s). A suitable vehicle is sterile saline. Other aqueous and non-aqueous isotonic sterile solutions and aqueous and non-aqueous sterile suspensions known to be pharmaceutically acceptable carriers and well known to those of skill in the art may be employed for this purpose. In some embodiments, as discussed in more detail below, an immunogenic composition is formulated with one or more food-grade components.

For example, in some embodiments, a lyophilized, lethally irradiated bacterial composition is stored in lyophilized form; then, just before use, the lyophilized, lethally-irradiated bacteria are solubilized, to generate a liquid formulation. In some embodiments, a lyophilized, lethally irradiated bacterial composition is formulated with other components (e.g., food-grade components), and is stored as a food product until use.

Optionally, an immunogenic composition of the invention may be formulated to contain other components, including, e.g., adjuvants, stabilizers, pH adjusters, preservatives and the like. Such components are well known to those of skill in the vaccine art. Suitable adjuvants include TLR ligands, as discussed above.

Suitable adjuvants include, but are not limited to, aluminum salt adjuvants (Nicklas (1992) Res. Immunol. 143:489-493); saponin adjuvants, e.g., QS21; Ribi's adjuvants (Ribi ImmunoChem Research Inc., Hamilton, Mont.); Montanide ISA adjuvants (e.g., ISA-51, ISA-57, ISA-720, ISA-151, etc.; Seppic, Paris, France); Hunter's TiterMax™ adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 143:553-557); alum (e.g., aluminum hydroxide, aluminum phosphate) emulsion based formulations including mineral oil, non-mineral oil, water-in-oil or oil-in-water emulsions, such as the Seppic ISA series of Montanide adjuvants; MF-59 (see, e.g., Granoff et al. (1997) Infect Immun. 65 (5):1710-1715); and PROVAX™ (IDEC Pharmaceuticals); poly[di(earboxylatophenoxy)phosphazene (PCPP) derivatives of lipopolysaccharides such as monophosphoryl lipid A (MPL®), muramyl dipeptide (MDP) and threonyl muramyl dipeptide (tMDP); OM-174 (a glucosamine disaccharide related to lipid A); Leishmania elongation factor; ISCOMS (immunostimulating complexes which contain mixed saponins and lipids, and form virus-sized particles with pores that can hold antigen); SB-AS2 (SB-AS2 (SmithKline Beecham adjuvant system #2; an oil-in-water emulsion containing MPL and QS21); SB-AS4 (SmithKline Beecham adjuvant system #4; contains alum and MPL); non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

In addition, other components that may modulate an immune response may be included in the formulation, including, but not limited to, cytokines, such as interleukins; colony-stimulating factors (e.g., GM-CSF, CSF, and the like); and tumor necrosis factor. Additional suitable additional components include toll-like receptor ligands, as discussed in more detail below.

In many embodiments, a subject composition is formulated for mucosal delivery. Mucosal delivery includes oral delivery; nasal delivery; delivery by inhalation (e.g. intranasal delivery, oral inhalational delivery, etc.); rectal delivery; and vaginal delivery. Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations for mucosal delivery are discussed in more detail below.

Subject lethally irradiated bacteria may be formulated for administration as suppositories. A low melting wax, such as a mixture of fatty acid glycerides or cocoa butter is first melted and the inactivated probiotic bacteria are dispersed homogeneously, for example, by stirring. The molten homogeneous mixture is then poured into conveniently sized molds, allowed to cool, and to solidify.

Subject lethally irradiated bacteria may be formulated for vaginal administration. Pessaries, tampons, creams, gels, pastes, foams or sprays, may contain agents in addition to the bacteria, such carriers, known in the art to be appropriate.

In some embodiments, a subject composition is formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., intradermal, topical application, intravenous, intramuscular, intratumoral, etc.

Irradiated Bacteria Formulated with a TLR Ligand

In some embodiments, a subject bacterial composition comprises lethally irradiated bacteria formulated with a toll-like receptor (TLR) ligand. In some particular embodiments, the TLR ligand is a TLR7 ligand. In other embodiments, the TLR ligand is a TLR8 ligand. In other particular embodiments, the TLR ligand is a TLR9 ligand. In still other particular embodiments, lethally irradiated bacteria are formulated with (and in some embodiments conjugated to) two or more different TLR ligands. In some embodiments, the TLR ligand is formulated in admixture with the lethally irradiated bacteria. In other embodiments, the TLR ligand is conjugated to the lethally irradiated bacteria.

Irradiated Bacteria Formulated in Admixture with a TLR Ligand

In some embodiments, a subject bacterial composition comprises lethally irradiated bacteria in admixture with at least one TLR ligand. In some embodiments, the TLR ligand is a monomeric TLR ligand. In other embodiments, the TLR ligand is multimerized. In many embodiments, the TLR ligand is synthetic. In many embodiments, the TLR ligand is pure. In many embodiments, the TLR ligand is a TLR agonist. In some embodiments, the TLR ligand is a chimeric TLR ligand.

In some embodiments, a subject bacterial composition comprises lethally irradiated bacteria in admixture with a TLR9 ligand. In some embodiments, a subject bacterial composition comprises lethally irradiated bacteria in admixture with a monomeric TLR7 ligand. In some embodiments, a subject bacterial composition comprises lethally irradiated bacteria in admixture with a monomeric TLR8 ligand. In some embodiments, a subject bacterial composition comprises lethally irradiated bacteria in admixture with a monomeric TLR9 ligand and a monomeric TLR8 ligand. In some embodiments, a subject bacterial composition comprises lethally irradiated bacteria in admixture with a monomeric TLR9 ligand and a monomeric TLR7 ligand. In some embodiments, a subject bacterial composition comprises lethally irradiated bacteria in admixture with a monomeric TLR7 ligand and a monomeric TLR8 ligand.

In some embodiments, a subject bacterial composition comprises lethally irradiated bacteria in admixture with a nucleic acid TLR9 ligand comprising a 5′-TCG-3′ sequence. In some embodiments, a subject bacterial composition comprises lethally irradiated bacteria in admixture with an imidazoquinoline TLR7 ligand. In some embodiments, a subject bacterial composition comprises lethally irradiated bacteria in admixture with a substituted guanine TLR7/8 ligand. In some embodiments, a subject bacterial composition comprises lethally irradiated bacteria in admixture with a TLR7 ligand selected from Loxoribine, 7-deazadeoxyguanosine, 7-thia-8-oxodeoxyguanosine, Imiquimod (R-837), and Resiquimod (R-848). In some embodiments, a subject bacterial composition comprises lethally irradiated bacteria in admixture with a nucleic acid TLR9 ligand comprising a 5′-TCG-3′ sequence and an imidazoquinoline TLR7 ligand. In some embodiments, a subject bacterial composition comprises lethally irradiated bacteria in admixture with a nucleic acid TLR9 ligand comprising a 5′-TCG-3′ sequence, and a substituted guanine TLR7/8 ligand. In some embodiments, a subject bacterial composition comprises lethally irradiated bacteria in admixture with a imidazoquinoline TLR7 ligand, and a substituted guanine TLR7/8 ligand. In some embodiments, a subject bacterial composition comprises lethally irradiated bacteria in admixture with a nucleic acid TLR9 ligand comprising a 5′-TCG-3′ sequence and a TLR7 ligand selected from Loxoribine, 7-deazadeoxyguanosine, 7-thia-8-oxodeoxyguanosine, Imiquimod (R-837), and Resiquimod (R-848).

In some embodiments, a subject bacterial composition comprises lethally irradiated bacteria in admixture with a multimeric TLR ligand. In some embodiments, a subject bacterial composition comprises lethally irradiated bacteria in admixture with a multimeric TLR9 ligand. In some embodiments, a subject bacterial composition comprises lethally irradiated bacteria in admixture with multimeric TLR7 ligand. In some embodiments, a subject bacterial composition comprises lethally irradiated bacteria in admixture with a multimeric TLR8 ligand.

In some embodiments, a subject bacterial composition comprises lethally irradiated bacteria in admixture with a chimeric TLR ligand. In some embodiments, a subject bacterial composition comprises lethally irradiated bacteria in admixture with a chimeric TLR ligand comprising a nucleic acid TLR9 ligand and a substituted guanine TLR7 ligand.

In some embodiments, any of the above-described subject bacterial compositions, comprising lethally irradiated bacteria in admixture with at least one TLR ligand, is modified to further include an additional adjuvant selected from an aluminum salt adjuvant, a saponin adjuvant (e.g., QS21), a Montanide ISA series adjuvant, MF-59, PROVAX™, MPL®, an ISCOM, SB-AS2, SB-AS4, PCPP, and TiterMax™.

In some embodiments, a subject bacterial composition comprises lethally irradiated bacteria in admixture with at least one TLR ligand, at a weight-based ratio of from about 10:1 to about 10¹⁰:1 irradiated bacteria:TLR ligand, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 lethally irradiated bacteria:TLR ligand. In some of these embodiments, the composition comprising lethally irradiated bacteria in admixture with at least one TLR ligand, further comprises an additional adjuvant selected from an aluminum salt adjuvant, a saponin adjuvant (e.g., QS21), a Montanide ISA series adjuvant, MF-59, PROVAX™, MPL®, an ISCOM, SB-AS2, SB-AS4, PCPP, and TiterMax™.

Irradiated Bacteria Conjugated with a TLR Ligand

In some embodiments, a subject bacterial composition comprises lethally irradiated bacteria conjugated to a TLR ligand. In some embodiments, a subject bacterial composition comprises lethally irradiated bacteria conjugated to a nucleic acid TLR9 ligand. In some embodiments, a subject bacterial composition comprises lethally irradiated bacteria conjugated to a monomeric TLR ligand. In other embodiments, a subject composition comprises lethally irradiated bacteria conjugated to a multimeric TLR ligand. In some embodiments, a subject bacterial composition comprises lethally irradiated bacteria conjugated to a monomeric TLR9 ligand. In some embodiments, a subject bacterial composition comprises lethally irradiated bacteria conjugated to a multimeric TLR9 ligand. In some embodiments, a subject bacterial composition comprises lethally irradiated bacteria conjugated to a monomeric TLR7 ligand. In some embodiments, a subject bacterial composition comprises lethally irradiated bacteria conjugated to a monomeric TLR8 ligand.

In some embodiments, a subject bacterial composition comprises lethally irradiated bacteria conjugated to a multimeric TLR ligand. In some embodiments, a subject bacterial composition comprises lethally irradiated bacteria conjugated to a multimeric nucleic acid TLR9 ligand.

In some embodiments, a subject bacterial composition comprises lethally irradiated bacteria conjugated to a chimeric TLR ligand. In some embodiments, a subject bacterial composition comprises irradiated bacteria conjugated to a chimeric TLR ligand that comprises a nucleic acid TLR9 ligand and a substituted guanine TLR7 ligand.

In some embodiments, a subject bacterial composition comprises irradiated bacteria conjugated to a nucleic acid TLR9. ligand comprising a 5′-TCG-3′ sequence.

Methods of conjugating a TLR ligand to a bacteria are well known in the art. For example, the chemistry for conjugating nucleic acids to proteins is well known in the art. The conditions for conjugation are such that the therapeutic effects of the bacteria are not substantially adversely affected.

TLR Ligands

A suitable TLR ligand is generally a TLR agonist. A TLR agonist is any compound or substance that functions to activate a TLR, e.g., to induce a signaling event mediated by a TLR signal transduction pathway. An example of a TLR ligand-mediated signal transduction event is activation of the IL-1 R-associated kinase, IRAK, and/or MAPK pathway, and/or NF-κB pathway and/or IRF pathway. Medzhitove et al. (1998) Mol. Cell 2:253-258; and Cao et al. (1996) Science 1128-1131. TLR include TLR1, TLR2, TLR3, TLR4, TLR5, TLR6, TLR7, TLR8, TLR9, and TLR10. Ozinsky et al. (2000) Proc. Natl. Acad Sci. USA 97:13766-13771; and Akira and Hemmi (2003) Immunol Lett. 85:85-95. TLR ligands include naturally-occurring TLR ligands, derivatives of naturally-occurring ligands, recombinant TLR ligands, and synthetic TLR ligands.

TLR1 functions in signaling as a dimer with TLR2. TLR1 agonists include, but are not limited to, tri-acylated lipopeptides, phenol-soluble modulin, lipopeptide from Mycobacterium tuberculosis, OSP A lipopeptide from Borrelia burgdorferi; and the like.

TLR2 ligands include, but are not limited to, bacterial or synthetic lipopetides, lipoproteins (including naturally-occurring lipoproteins; derivatives of naturally-occurring lipoproteins; synthetic lipoproteins); lipopeptides (Takeuchi et al. (2000) J. Immunol. 164:554-557), e.g., lipopeptides from Mycobacteria tuberculosis, Borrelia burgdorferi, Treponema pallidum, etc.; whole bacteria, e.g., heat-killed Acholeplasma laidlawii, heat-killed Listeria monocytogenes (Flo et al. (2000) J. Immunol. 164:2064-2069), and the like; lipoteichoic acids (Schwandner et al. (1999) J. Biol. Chem. 274:17406-17409); peptidoglycans (Takeuchi et al. (1999) Immunity 11:443-451), e.g., peptidoglycans from Staphylococcus aureus, etc.; mannuronic acids; Neisseria porins; bacterial fimbriae, Yersinia virulence factors, cytomegalovirus virions, measles haemagglutinin; yeast cell wall extracts; yeast particle zymosan; glycosyl phosphatidyl inositol (GPI) anchor from Trypanosoma cruzi; and the like.

TLR2 agonists include synthetic triacylated and diacylated lipopeptides. An exemplary, non-limiting TLR2 ligand is Pam₃Cys (tripalmitoyl-S-glyceryl cysteine). Aliprantis et al. (1999) Science 285:736-739. Derivatives of Pam₃Cys are also suitable TLR2 agonists, where derivatives include, but are not limited to, Pam₃Cys-Ser-Ser-Asn-Ala; Pam₃Cys-Ser-(Lys)₄; Pam3Cys-Ala-Gly; Pam3Cys-Ser-Gly; Pam3Cys-Ser; Pam3Cys-OMe; Pam3Cys-OH; PamCAG, palmitoyl-Cys((RS)-2,3-di(palmitoyloxy)-propyl)-Ala-Gly-OH; and the like. Another non-limiting example of a suitable TLR2 agonist is PAM2CSK4. PAM2CSK4 (dipalmitoyl-S-glyceryl cysteine-serine-(lysine)₄; or Pam2Cys-Ser-(Lys)₄) is a synthetic diacylated lipopeptide. Synthetic TLRs agonists have been described in the literature. See, e.g., Kellner et al. (1992) Biol Chem Hoppe Seyler 373:1:51-5; Seifer et al. (1990) Biochem. J. 26:795-802; Lee et al. (2003) Journal of Lipid Research 44:479-486.

TLR3 ligands include naturally-occurring double-stranded RNA (dsRNA); synthetic ds RNA; and synthetic dsRNA analogs; and the like. Alexopoulou et al. (2001) Nature 413:732-738. An exemplary, non-limiting example of a synthetic ds RNA analog is poly(I:C).

TLR4 ligands include naturally-occurring lipopolysaccharides (LPS), e.g., LPS from a wide variety of Gram negative bacteria; derivatives of naturally-occurring LPS; synthetic LPS; bacteria heat shock protein-60 (Hsp60); mannuronic acid polymers; flavolipins; teichuronic acids; S. pneumoniae pneumolysin; bacterial fimbriae, respiratory syncytial virus coat protein; and the like.

TLR5 ligands include flagellin, e.g., naturally-occurring flagellin, recombinant flagellin, synthetic flagellin, flagellin fragments; and the like.

TLR 6 ligands include mycoplasma lipoproteins; lipoteichoic acid; bacterial peptidoglycans; di-acylated lipopeptides; peptidoglycan; phenol-soluble modulin; and the like.

TLR7 ligands include imidazoquinoline compounds; guanosine analogs; pyrimidinone compounds such as bropirimine and bropirimine analogs; and the like. Imidazoquinoline compounds that function as TLR7 ligands include, but are not limited to, imiquimod, (also known as Aldara, R-837, S-26308), and R-848 (also known as resiquimod, S-28463). Suitable imidazoquinoline agents include imidazoquinoline amines, imidazopyridine amines, 6,7-fused cycloalkylimidazopyridine amines, and 1,2 bridged imidazoquinoline amines. These compounds have been described in U.S. Pat. Nos. 4,689,338, 4,929,624, 5,238,944, 5,266,575, 5,268,376, 5,346,905, 5,352,784, 5,389,640, 5,395,937, 5,494,916, 5,482,936, 5,525,612, 6,039,969 and 6,110,929. Particular species of imidazoquinoline agents include R-848 (S-28463); 4-amino-2ethoxymethyl-α,α-dimethyl-1H-imidazo[4,5-c]quinoline-s-1-ethanol; and 1-(2-methylpropyl)-1H-imidazo[4,5-c]quinolin-4-amine (R-837 or Imiquimod). Guanosine analogs that function as TLR7 ligands include certain C8-substitutes and N7,C8-disubstituted guanine ribonucleotides and deoxyribonucleotides, including, but not limited to, Loxoribine (7-allyl-8-oxoguanosine), 7-thia-8-oxo-guanosine (TOG), 7-deazaguanosine, and 7-deazadeoxyguanosine. Lee et al. (2003) Proc. Natl. Acad. Sci. USA 100:6646-6651. Bropirimine (PNU-54461), a 5-halo-6-phenyl-pyrimidinone, and bropirimine analogs are described in the literature and are also suitable for use. See, e.g., Vroegop et al. (1999) Intl. J. Immunopharmacol. 21:647-662. Additional examples of suitable C8-substituted guanosines include but are not limited to 8-mercaptoguanosine, 8-bromoguanosine, 8-methylguanosine, 8-oxo-7,8-dihydroguanosine, C8-arylamino-2′-deoxyguanosine, C8-propynyl-guanosine, C8- and N7-substituted guanine ribonucleosides such as 7-allyl-8-oxoguanosine (loxoribine) and 7-methyl-8-oxoguanosine, 8-aminoguanosine, 8-hydroxy-2′-deoxyguanosine, and 8-hydroxyguanosine.

In some embodiments a substituted guanine TLR7 ligand is monomeric. In other embodiments, a substituted guanine TLR7 ligand is multimeric. Thus, in some embodiments, a TLR7 ligand has the formula: (B)_{q}, where B is a substituted guanine TLR7 ligand, and q is 1, 2, 3, 4, 5 6, 7, 8, 9, or 10. The individual TLR7 ligand monomers in a multimeric TLR7 ligand are linked, covalently or non-covalently, either directly to one another or through a linker.

TLR8 ligands include, but are not limited to, compounds such as R-848, and derivatives and analogs thereof.

TLR 9 Ligands

Examples of TLR9 ligands include nucleic acids comprising the sequence 5′-CG-3′, particularly where the C is unmethylated. The terms “polynucleotide,” and “nucleic acid,” as used interchangeably herein in the context of TLR9 ligand molecules, refer to a polynucleotide of any length, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus a TLR9 ligand may be, for example, single-stranded DNA (ssDNA), double-stranded DNA (dsDNA), single-stranded RNA (ssRNA) or double-stranded RNA (dsRNA). TLR9 ligands also encompasses crude, detoxified bacterial (e.g., mycobacterial) RNA or DNA, as well as enriched plasmids enriched for a TLR9 ligand. In some embodiments, a “TLR9 ligand-enriched plasmid” refers to a linear or circular plasmid that comprises or is engineered to comprise a greater number of CpG motifs than normally found in mammalian DNA.

Exemplary, non-limiting TLR9 ligand-enriched plasmids are described in, for example, Roman et al. (1997) Nat Med. 3(8):849-54. Modifications of oligonucleotides include, but are not limited to, modifications of the 3′OH or 5′OH group, modifications of the nucleotide base, modifications of the sugar component, and modifications of the phosphate group.

A TLR9 ligand may comprise at least one nucleoside comprising an L-sugar. The L-sugar may be deoxyribose, ribose, pentose, deoxypentose, hexose, deoxyhexose, glucose, galactose, arabinose, xylose, lyxose, or a sugar “analog” cyclopentyl group.

The L-sugar may be in pyranosyl or furanosyl form.

TLR9 ligands generally do not provide for, nor is there any requirement that they provide for, expression of any amino acid sequence encoded by the polynucleotide, and thus the sequence of a TLR9 ligand may be, and generally is, non-coding. TLR9 ligands may comprise a linear double or single-stranded molecule, a circular molecule, or can comprise both linear and circular segments. TLR9 ligands may be single-stranded, or may be completely or partially double-stranded.

In some embodiments, a TLR9 ligand for use in a subject method is an oligonucleotide, e.g., consists of a sequence of from about 5 nucleotides to about 200 nucleotides, from about 10 nucleotides to about 100 nucleotides, from about 12 nucleotides to about 50 nucleotides, from about 15 nucleotides to about 25 nucleotides, from 20 nucleotides to about 30 nucleotides, from about 5 nucleotides to about 15 nucleotides, from about 5 nucleotides to about 10 nucleotides, or from about 5 nucleotides to about 7 nucleotides in length. In some embodiments, a TLR9 ligand that is less than about 15 nucleotides, less than about 12 nucleotides, less than about 10 nucleotides, or less than about 8 nucleotides in length is associated with a larger molecule, e.g., adsorbed onto an insoluble support, as described below.

In some embodiments, a TLR9 ligand does not provide for expression of a peptide or polypeptide in a eukaryotic cell, e.g., introduction of a TLR9 ligand into a eukaryotic cell does not result in production of a peptide or polypeptide, because the TLR9 ligand does not provide for transcription of an mRNA encoding a peptide or polypeptide. In these embodiments, a TLR9 ligand lacks promoter regions and other control elements necessary for transcription in a eukaryotic cell.

A TLR9 ligand can be isolated from a bacterium, e.g., separated from a bacterial source; produced by synthetic means (e.g., produced by standard methods for chemical synthesis of polynucleotides); produced by standard recombinant methods, then isolated from a bacterial source; or a combination of the foregoing. In many embodiments, a TLR9 ligand is purified, e.g., is at least about 80%, at least about 90%, at least about 95%, at least about 98%, at least about 99%, or more, e.g., 99.5%, 99.9%, or more, pure.

In other embodiments, a TLR9 ligand is part of a larger nucleotide construct (e.g., a plasmid vector, a viral vector, or other such construct). A wide variety of plasmid and viral vector are known in the art, and need not be elaborated upon here. A large number of such vectors has been described in various publications, including, e.g., Current Protocols in Molecular Biology, (F. M. Ausubel, et al., Eds. 1987, and updates). Many vectors are commercially available.

In general, a TLR9 ligand used in a subject composition comprises at least one unmethylated CpG motif. The relative position of any CpG sequence in a polynucleotide in certain mammalian species (e.g., rodents) is 5′-CG-3′ (i.e., the C is in the 5′ position with respect to the G in the 3′ position).

In some embodiments, a TLR9 ligand comprises a central palindromic core sequence comprising at least one CpG sequence, where the central palindromic core sequence contains a phosphodiester backbone, and where the central palindromic core sequence is flanked on one or both sides by phosphorothioate backbone-containing polyguanosine sequences.

In other embodiments, a TLR9 ligand comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the TLR9 ligand comprises (TCG)n, where n=one to three, at the 5′ end of the nucleic acid. In other embodiments, the TLR9 ligand comprises (TCG)n, where n=one to three, and where the (TCG)n sequence is flanked by one nucleotide, two nucleotides, three nucleotides, four nucleotides, or five nucleotides, on the 5′ end of the (TCG)n sequence.

Exemplary consensus CpG motifs of TLR9 ligands useful in the invention include, but are not necessarily limited to:
5′-Purine-Purine-(C)-(G)-Pyrimidine-Pyrimidine-3′, in which the TLR9 ligand comprises a CpG motif flanked by at least two purine nucleotides (e.g., GG, GA, AG, AA, II, etc.,) and at least two pyrimidine nucleotides (CC, TT, CT, TC, UU, etc.);
5′-Purine-TCG-Pyrimidine-Pyrimidine-3′;
5′-TCG-N-N-3′; where N is any base;
5′-Nₓ(CG)ₙN_{y}, where N is any base, where x and y are independently any integer. from 0 to 200, e.g., 0, 1, 2, 3, 4, 5, 6, 7, 8, 9, 10, 11-15, 16-20, 21-25, 25-30, 30-50, 50-75, 75-100, 100-150, or 1 50-200; and n is any integer that is 1 or greater, e.g., 1, 2, 3, 4, 5, 6, 7, 8, 9, 10, or greater.
5′-Nₓ(TCG)ₙN_{y}, where N is any base, where x and y are independently any integer from 0 to 200, e.g., 0, 1, 2, 3, 4, 5, 6, 7, 8, 9, 10, 11-15, 16-20, 21-25, 25-30, 30-50, 50-75, 75-100, 100-150, or 150-200; and n is any integer that is 1 or greater, e.g., 1, 2, 3, 4, 5, 6, 7, 8, 9, 10, or greater.
5′-(TCG)ₙ-3′, where n is any integer that is 1 or greater, e.g., to provide a TCG-based TLR9 ligand (e.g., where n=3, the polynucleotide comprises the sequence 5′-TCGNNTCGNNTCG-3′; SEQ ID NO:1);
5′Nₘ-(TCG)ₙ-Nₚ-3′, where N is any nucleotide, where m is zero, one, two, or three, where n is any integer that is 1 or greater, and where p is one, two, three, or four;
5′Nₘ-(TCG)ₙ-Nₚ-3′, where N is any nucleotide, where m is zero to 5, and where n is any integer that is 1 or greater, where p is four or greater, and where the sequence N—N—N—N comprises at least two CG dinucleotides that are either contiguous with each other or are separated by one nucleotide, two nucleotides, or three nucleotides; and
5′-Purine-Purine -CG-Pyrimidine-TCG-3′.

A non-limiting example of a TLR9 ligand comprising 5′-(TCG)ₙ-3′, where n is any integer that is 1 or greater, is a TLR9 ligand comprising the sequence 5′ TCGTCGTTTTGTCGTTTTGTCGTT 3′ (SEQ ID NO:2).

Where a nucleic acid TLR9 ligand comprises a sequence of the formula: 5′-Nₘ-(TCG)ₙ-Nₚ-3′, where N is any nucleotide, where m is zero to 5, and where n is any integer that is 1 or greater, where p is four or greater, and where the sequence N—N—N—N comprises at least two CG dinucleotides that are either contiguous with each other or are separated by one nucleotide, two nucleotides, or three nucleotides, exemplary TLR9 ligands useful in the invention include, but are not necessarily limited to:
(1) a sequence of the formula in which n=2, and Nₚ is NNCGNNCG;
(2) a sequence of the formula in which n=2, and Nₚ is AACGTTCG;
(3) a sequence of the formula in which n=2, and Nₚ is TTCGAACG;
(4) a sequence of the formula in which n=2, and Nₚ is TACGTACG;
(5) a sequence of the formula in which n=2, and Nₚ is ATCGATCG;
(6) a sequence of the formula in which n=2, and Nₚ is CGCGCGCG;
(7) a sequence of the formula in which n=2, and Nₚ is GCCGGCCG;
(8) a sequence of the formula in which n=2, and Nₚ is CCCGGGCG;
(9) a sequence of the formula in which n=2, and Nₚ is GGCGCCCG;
(10) a sequence of the formula in which n=2, and Nₚ is CCCGTTCG;
(11) a sequence of the formula in which n=2, and Nₚ is GGCGTTCG;
(12) a sequence of the formula in which n=2, and Nₚ is TTCGCCCG;
(13) a sequence of the formula in which n=2, and Nₚ is TTCGGGCG;
(14) a sequence of the formula in which n=2, and Nₚ is AACGCCCG;
(15) a sequence of the formula in which n=2, and Nₚ is AACGGGCG;
(16) a sequence of the formula in which n=2, and Nₚ is CCCGAACG; and
(17) a sequence of the formula in which n=2, and Nₚ is GGCGAACG;
and where, in any of 1-17, m=zero, one, two, or three.

Where a nucleic acid TLR9 ligand comprises a sequence of the formula: 5′ Nₘ-(TCG)ₙ-Nₚ-3′, where N is any nucleotide, where m is zero, one, two, or three, where n is any integer that is 1 or greater, and where p is one, two, three, or four, exemplary TLR9 ligands useful in the invention include, but are not necessarily limited to:
(1) a sequence of the formula where m=zero, n=1, and Nₚ is T-T-T;
(2) a sequence of the formula where m=zero, n=1, and Nₚ is T-T-T-T;
(3) a sequence of the formula where m=zero, n=1, and Nₚ is C-C-C-C;
(4) a sequence of the formula where m=zero, n=1, and Nₚ is A-A-A-A;
(5) a sequence of the formula where m=zero, n=1, and Nₚ is A-G-A-T;
(6) a sequence of the formula where Nₘ is T, n=1, and Nₚ is T-T-T;
(7) a sequence of the formula where Nₘ is A, n=1, and Nₚ is T-T-T;
(8) a sequence of the formula where Nₘ is C, n=1, and Nₚ is T-T-T;
(9) a sequence of the formula where Nₘ is G, n=1, and Nₚ is T-T-T;
(10) a sequence of the formula where Nₘ is T, n=1, and Nₚ is A-T-T;
(11) a sequence of the formula where Nₘ is A, n=1, and Nₚ is A-T-T; and
(12) a sequence of the formula where Nₘ is C, n=1, and Nₚ is A-T-T.

The core structure of a TLR9 ligand useful in the invention may be flanked upstream and/or downstream by any number or composition of nucleotides or nucleosides. In some embodiments, the core sequence of a TLR9 ligand is at least 6 bases or 8 bases in length, and the complete TLR9 ligand (core sequences plus flanking sequences 5′, 3′ or both) is usually between 6 bases or 8 bases, and up to about 200 bases in length.

Exemplary DNA-based TLR9 ligands useful in the invention include, but are not necessarily limited to, polynucleotides comprising one or more of the following nucleotide sequences:

AGCGCT, AGCGCC, AGCGTT, AGCGTC, AACGCT, AACGCC,
AACGTT, AACGTC, GGCGCT, GGCGCC, GGCGTT, GGCGTC,
GACGCT, GACGCC, GACGTT, GACGTC, GTCGTC, GTCGCT,
GTCGTT, GTCGCC, ATCGTC, ATCGCT, ATCGTT, ATCGCC,
TCGTCG,
and
TCGTCGTCG.

Additional exemplary TLR9 ligands useful in the invention include, but are not necessarily limited to, polynucleotides comprising one or more of the following nucleotide sequences:

TCGXXXX, TCGAXXX, XTCGXXX, XTCGAXX, TCGTCGA,
TCGACGT, TCGAACG, TCGAGAT, TCGACTC, TCGAGCG,
TCGATTT, TCGCTTT, TCGGTTT, TCGTTTT, TCGTCGT,
ATCGATT, TTCGTTT, TTCGATT, ACGTTCG, AACGTTC,
TGACGTT, TGTCGTT, TCGXXX, TCGAXX, TCGTCG,
AACGTT, ATCGAT, GTCGTT, GACGTT, TCGXX, TCGAX,
TCGAT, TCGTT, TCGTC, TCGA, TCGT, TCGX,
and
TCG
(where “X” is any nucleotide).

Exemplary DNA-based TLR9 ligands useful in the invention include, but are not necessarily limited to, polynucleotides comprising the following octameric nucleotide sequences:

AGCGCTCG, AGCGCCCG, AGCGTTCG, AGCGTCCG, AACGCTCG,
AACGCCCG, AACGTTCG, AACGTCCG, GGCGCTCG, GGCGCCCG,
GGCGTTCG, GGCGTCCG, GACGCTCG, GACGCCCG, GACGTTCG,
and
GACGTCCG.

A TLR9 ligand useful in carrying out a subject method can comprise one or more of any of the above CpG motifs. For example, a TLR9 ligand useful in the invention can comprise a single instance or multiple instances (e.g., 2, 3, 4, 5 or more) of the same CpG motif. Alternatively, a TLR9 ligand can comprise multiple CpG motifs (e.g., 2, 3, 4, 5 or more) where at least two of the multiple CpG motifs have different consensus sequences, or where all CpG motifs in the TLR9 ligand have different consensus sequences.

A TLR9 ligand useful in the invention may or may not include palindromic regions. If present, a palindrome may extend only to a CpG motif, if present, in the core hexamer or octamer sequence, or may encompass more of the hexamer or octamer sequence as well as flanking nucleotide sequences.

Multimeric TLR9 Ligands

In some embodiments, a TLR9 ligand is multimeric. A multimeric TLR9 ligand comprises two, three, four, five, six, seven, eight, nine, ten, or more individual (monomeric) nucleic acid TLR9 ligands, as described above, linked via non-covalent bonds, linked via covalent bonds, and either linked directly to one another, or linked via one or more spacers. Suitable spacers include nucleic acid and non-nucleic acid molecules, as long as they are biocompatible. In some embodiments, multimeric TLR9 ligand comprises a linear array of monomeric TLR9 ligands. In other embodiments, a multimeric TLR9 ligand is a branched, or dendrimeric, array of monomeric TLR9 ligands.

Multimeric TLR9 ligand complexes can be formed with non-covalent interactions, such as ionic bonds, hydrophobic interactions, hydrogen bonds and/or van der Waals attractions. For example, a multimeric TLR9 ligand can be a non-covalently linked aggregate of monomeric TLR9 ligands.

In some embodiments, a multimeric TLR9 ligand forms aggregates in vivo and/or in vitro. In some embodiments, a multimeric TLR9 ligand forms a secondary structure(s) near the core CpG motifs. In some embodiments, a multimeric TLR9 ligand comprises both a multimerization domain and a receptor binding CpG domain, which multimerization domain and receptor binding CpG domain are spatially distinct.

In some embodiments, a multimeric TLR9 ligand has the general structure Xₙ, where X is a nucleic acid TLR9 ligand as described above, and having a length of from about 6 nucleotides to about 200 nucleotides, e.g., from about 6 nucleotides to about 8 nucleotides, from about 8 nucleotides to about 10 nucleotides, from about 10 nucleotides to about 12 nucleotides, from about 12 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, from about 50 nucleotides to about 60 nucleotides, from about 60 nucleotides to about 70 nucleotides, from about 70 nucleotides to about 80 nucleotides, from about 80 nucleotides to about 90 nucleotides, from about 90 nucleotides to about 100 nucleotides, from about 100 nucleotides to about 125 nucleotides, from about 125 nucleotides to about 150 nucleotides, from about 150 nucleotides to about 175 nucleotides, or from about 175 nucleotides to about 200 nucleotides; and where n is any number from one to about 100, e.g., n=1,2, 3,4, 5, 6, 7, 8,9, 10, from 10 to about 15, from 15 to about 20, from 20 to about 25, from 25 to about 30, from 30 to about 40, from 40 to about 50, from 50 to about 60, from 60 to about 70, from 70 to about 80, from 80 to about 90, or from 90 to about 100.

In some embodiments, a multimeric TLR9 ligand has the general structure (X₁)ₙ(X₂)ₙ where X is a nucleic acid TLR9 ligand as described above, and having a length of from about 6 nucleotides to about 200 nucleotides, e.g., from about 6 nucleotides to about 8 nucleotides, from about 8 nucleotides to about 10 nucleotides, from about 10 nucleotides to about 12 nucleotides, from about 12 nucleotides to about 15 nucleotides, from about 15 nucleotides to about 20 nucleotides, from about 20 nucleotides to about 25 nucleotides, from about 25 nucleotides to about 30 nucleotides, from about 30 nucleotides to about 40 nucleotides, from about 40 nucleotides to about 50 nucleotides, from about 50 nucleotides to about 60 nucleotides, from about 60 nucleotides to about 70 nucleotides, from about 70 nucleotides to about 80 nucleotides, from about 80 nucleotides to about 90 nucleotides, from about 90 nucleotides to about 100 nucleotides, from about 100 nucleotides to about 125 nucleotides, from about 125 nucleotides to about 150 nucleotides, from about 150 nucleotides to about 175 nucleotides, or from about 175 nucleotides to about 200 nucleotides; and where n is any number from one to about 100, e.g., n=1, 2, 3, 4, 5, 6, 7, 8, 9, 10, from 10 to about 15, from 15 to about 20, from 20 to about 25, from 25 to about 30, from 30 to about 40, from 40 to about 50, from 50 to about 60, from 60 to about 70, from 70 to about 80, from 80 to about 90, or from 90 to about 100. In these embodiments, X₁ and X₂ differ in nucleotide sequence from one another by at least one nucleotide, and may differ in nucleotide sequence from one another by two, three, four, five, six, seven, eight, nine, ten, or more bases. In some of these embodiments, the multimeric nucleic acid TLR9 ligand includes further (Xₐ)ₙ, where each Xₐ is a monomeric TLR9 ligand as defined above, n=0, 1, 2, 3, 4, 5, 6, 7, 8, 9, 10, from 10 to about 15, from 15 to about 20, from 20 to about 25, from 25 to about 30, from 30 to about 40, from 40 to about 50, from 50 to about 60, from 60 to about 70, from 70 to about 80, from 80 to about 90, or from 90 to about 100, and where Xₐ=X₃, X₃X₄, X₃X₄X₅, X₃X₄X₅X₆, etc., and where each of X of X₃, X₃X₄, X₃X₄X₅, X₃X₄X₅X₆, etc. has the same or different nucleotide sequence from X₁ and/or X₂.

As noted above, in some embodiments, a subject multimeric TLR9 ligand comprises a. TLR9 ligand separated from an adjacent TLR9 ligand by a spacer. In some embodiments, a spacer is a non-TLR9 ligand nucleic acid. In other embodiments, a spacer is a non-nucleic acid moiety. Suitable spacers include those described in U.S. patent publication No. 20030225016.

A TLR9 ligand is multimerized using any known method. In some embodiments, a nucleic acid TLR9 ligand is multimerized using a method as described in Example 2, below.

In some embodiments, a nucleic acid TLR9 ligand comprises a guanine-rich 3′ tail. The presence of a guanine-rich 3′ tail promotes multimerization of a nucleic acid TLR9 ligand. A guanine-rich 3′ tail can comprise from about 4 guanine residues to about 50 guanine residues, e.g., from about 4 guanine residues to about 6 guanine residues, from about 6 guanine residues to about 10 guanine residues, from about 10 guanine residues to about 15 guanine residues, from about 15 guanine residues to about 20 guanine residues, from about 20 guanine residues to about 25 guanine residues, from about 25 guanine residues to about 50 guanine residues, from about 50 guanine residues to about 75 guanine residues, or from about 75 guanine residues to about 100 guanine residues, in a guanine-rich tail having a length of from about 4 nucleotides to about 200 nucleotides, e.g., from about 4 nucleotides to about 10 nucleotides, from about 10 nucleotides to about 20 nucleotides, from about 20 nucleotides to about 50 nucleotides, from about 50 nucleotides to about 100 nucleotides, or from about 100 nucleotides to about 200 nucleotides. Typically, the proportion of guanine residues in a guanine-rich tail ranges from about 30% to about 100%, e.g., from about 30% to about 40%, from about 40% to about 50%, from about 50% to about 60%, from about 60% to about 70%, from about 70% to about 80%, from about 80% to about 90%, or from about 90% to about 100%.

In some embodiments, a TLR9 ligand comprises the sequence 5′-Xₙ-CG-Xₘ-(A)-3′, where A is a guanine-rich tail as described above, where X is any nucleotide, and n and m are independently an integer from 0 to 200. In some embodiments, a TLR9 ligand comprises the sequence 5′-Xₙ-(TCG)ₚ-Xₘ-(A)-3′, where A is a guanine-rich tail as described above, where X is any nucleotide, n and m are independently an integer from 0 to 200, and p is 1, 2, 3, 4, 5, 6, 7, 8, 9, or 10.

Chimeric TLR Ligands

In some embodiments, a TLR ligand is a chimeric TLR ligand. In some embodiments, a chimeric TLR ligand comprises a nucleic acid TLR9 ligand moiety, and a substituted guanine TLR7 ligand moiety.

In some embodiments, a chimeric TLR ligand has the following formula: 5′-Xₙ-CG-Xₘ-(B)_{q}-3′, where X is any nucleotide, and n and m are independently an integer from 0 to 200, and where B is a substituted guanine TLR7 ligand, and q is 1, 2, 3, 4, 5, 6, 7, 8, 9, or 10.

In some embodiments, a chimeric TLR ligand has the following formula: 5′-Xₙ-(TCG)ₚ-Xₘ-(B)_{q}-3′, where X is any nucleotide, n and m are each independently an integer from 0 to 200, where B is a substituted guanine TLR7 ligand, and where q and p are each independently 1, 2, 3, 4, 5, 6, 7, 8, 9, or 10.

In some embodiments, a chimeric TLR ligand has the following formula: 5′-(B)_{q}-Xₙ-CG-Xₘ-3′, where X is any nucleotide, and n and m are independently an integer from 0 to 200, where B is a substituted guanine TLR7 ligand, and q is 1, 2, 3, 4, 5, 6, 7, 8, 9, or 10.

In some embodiments, a chimeric TLR ligand has the following formula: 5′-(B)_{q}-Xₙ-(TCG)ₚ-Xₘ-3′, where X is any nucleotide, n and m are each independently an integer from 0 to 200, and q and p are each independently 1, 2, 3, 4, 5, 6, 7, 8, 9, or 10.

TLR9 Ligand Modifications

A TLR9 ligand suitable for use in a subject composition can be modified in a variety of ways. For example, a TLR9 ligand can comprise backbone phosphate group modifications (e.g., methylphosphonate, phosphorothioate, phosphoroamidate and phosphorodithioate internucleotide linkages), which modifications can, for example, enhance their stability in vivo, making them particularly useful in therapeutic applications. A particularly useful phosphate group modification is the conversion to the phosphorothioate or phosphorodithioate forms of a nucleic acid TLR9 ligand. Phosphorothioates and phosphorodithioates are more resistant to degradation in vivo than their unmodified oligonucleotide counterparts, increasing the half-lives of the TLR9 ligands and making them more available to the subject being treated.

Other modified TLR9 ligands encompassed by the present invention include TLR9 ligands having modifications at the 5′ end, the 3′ end, or both the 5′ and 3′ ends. For example, the 5′ and/or 3′ end can be covalently or non-covalently associated with a molecule (either nucleic acid, non-nucleic acid, or both) to, for example, increase the bio-availability of the TLR9 ligand, increase the efficiency of uptake where desirable, facilitate delivery to cells of interest, and the like. Exemplary molecules for conjugation to a TLR9 ligand include, but are not necessarily limited to, cholesterol, phospholipids, fatty acids, sterols, oligosaccharides, polypeptides (e.g., immunoglobulins), peptides, antigens (e.g., peptides, small molecules, etc.), linear or circular nucleic acid molecules (e.g., a plasmid), insoluble supports, and the like.

A TLR9 ligand is in some embodiments linked (e.g., conjugated, covalently linked, non-covalently associated with, or adsorbed onto) an insoluble support. An exemplary, non-limiting example of an insoluble support is cationic poly(D,L-lactide-co-glycolide).

Additional TLR9 ligand conjugates, and methods for making same, are known in the art and described in, for example, WO 98/16427 and WO 98/55495. Thus, the term TLR9 ligand” includes conjugates comprising a nucleic acid TLR9 ligand.

A polypeptide, e.g., a therapeutic polypeptide, may be conjugated directly or indirectly, e.g., via a linker molecule, to a TLR9 ligand. A wide variety of linker molecules are known in the art and can be used in the conjugates. The linkage from the peptide to the oligonucleotide may be through a peptide reactive side chain, or the N— or C-terminus of the peptide. Linkage from the oligonucleotide to the peptide may be at either the 3′ or 5′ terminus, or internal. A linker may be an organic, inorganic, or semi-organic molecule, and may be a polymer of an organic molecule, an inorganic molecule, or a co-polymer comprising both inorganic and organic molecules.

If present, the linker molecules are generally of sufficient length to permit oligonucleotides and/or polynucleotides and a linked polypeptide to allow some flexible movement between the oligonucleotide and the polypeptide. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to oligonucleotides may be used in light of this disclosure.

Peptides may be synthesized chemically or enzymatically, may be produced recombinantly, may be isolated from a natural source, or a combination of the foregoing. Peptides may be isolated from natural sources using standard methods of protein purification known in the art, including, but not limited to, HPLC, exclusion chromatography, gel electrophoresis, affinity chromatography, fast protein liquid chromatography, or other purification technique. One may employ solid phase peptide synthesis techniques, where such techniques are known to those of skill in the art. See Jones, The Chemical Synthesis of Peptides (Clarendon Press, Oxford)(1994). Generally, in such methods a peptide is produced through the sequential additional of activated monomeric units to a solid phase bound growing peptide chain. Well-established recombinant DNA techniques can be employed for production of peptides.

The present invention provides TLR ligand compositions comprising a multimeric TLR9 ligand; TLR ligand compositions comprising a multimeric TLR7 ligand, particularly a substituted guanine multimeric TLR7 ligand; and TLR ligand compositions comprising a chimeric TLR ligand. In some embodiments, the TLR ligand compositions are pharmaceutical compositions comprising a pharmaceutically acceptable excipient. In some embodiments, a subject TLR ligand composition further comprises an antigen (e.g., a microbial antigen, an allergen, a tumor-associated antigen). In some embodiments, a subject TLR ligand composition further comprises an adjuvant. In some embodiments, the adjuvant is selected from selected from an aluminum salt adjuvant, a saponin adjuvant (e.g., QS21), a Montanide ISA series adjuvant, MF-59, PROVAX™, MPL®, an ISCOM, SB-AS2, SB-AS4, PCPP, and TiterMax™.

TLR ligand compositions are formulated as discussed herein. Additional formulations are well known in the art. A subject TLR ligand composition is useful for inducing a Th1-type immune response (and reducing a Th2-type immune response) in an individual. Thus, a subject TLR ligand composition is useful for treating conditions amenable to treatment by inducing a Th1 response in an individual.

The present invention provides methods of inducing a Th1 immune response in an individual to an antigen, the method comprising administering an effective amount of a subject TLR ligand composition, where the TLR ligand composition comprises the antigen. Methods of inducing an immune response, particularly a Th1 immune response, in an individual to an antigen are useful for inducing protective immunity to a microbial pathogen (e.g., where the antigen is a microbial antigen); for reducing tumor load (e.g., where the antigen is a TAA); and for treating an allergy (e.g., where the antigen is an allergen).

A subject TLR ligand composition is of particular use in stimulating the Th1 compartment in preference to the Th2 compartment, thus suppressing IgE production in response to an antigen. Thus, the invention further provides methods for reducing IgE production in response to an antigen, generally involving administering a subject TLR ligand composition to an individual sensitized to the antigen. The invention provides methods of treating an allergy in an individual sensitized to an allergen, generally involving administering to the individual a subject TLR ligand composition comprising an effective amount of the allergen to which the individual is sensitized.

Nutraceutical Formulations: Food Products

The present invention provides nutraceutical formulations comprising subject lethally irradiated bacteria; and food products comprising lethally irradiated bacteria.

The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Thus, by definition, pharmaceutical compositions comprising a subject bacterial composition include nutraceuticals. Also by definition, pharmaceutical compositions comprising a subject bacterial composition include compositions comprising a subject bacterial composition and a food-grade component. In some embodiments, a subject lethally irradiated bacterial composition is added to food products to provide a health benefit, e.g., to induce an immune response to a pathogenic microorganism.

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candy; and the like. These foods are modified by the inclusion of a subject bacterial composition. For example, to induce an immune response to a pathogenic bacterium, a subject food formulation comprising a subject bacterial composition is ingested once, or more than once, e.g., once per week, once daily, or some other interval.

The present invention provides compositions (e.g., nutraceutical compositions) comprising a subject bacterial composition and a food-grade pharmaceutically acceptable excipient. In many embodiments, a subject bacterial nutraceutical composition includes one or more components found in food products. Thus, the instant invention provides a food composition and products comprising a subject bacterial composition and a food component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. Other suitable components include soy-based components. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides non-digestible carbohydrates are resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, diacetyl tartaric acid esters of mono-diglycerides (DATEM), citric acid esters of monoglycerides (CITREM) and combinations thereof. Additional suitable emulsifiers include DIMODAN, including DIMODAN™ B 727 and DIMODAN™ PV, GRINDSTED™ CITREM, GRINDSTED™ GA, GRINDSTED™ PS such as GRINDSTED™ PS 100, GRINDSTED™ PS 200, GRINDSTED™ PS 300, GRINDSTED™ PS 400; RYLO™ (manufactured and distributed by DANISCO CULTOR), including RYLO™ AC, RYLO™ CI, RYLO™ LA, RYLO™ MD, RYLO™ MG, RYLO™ PG, RYLO™ PR, RYLO™ SL, RYLO™ SO, RYLO™ TG; and combinations thereof.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2, red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

Lethally irradiated bacteria are present in the food product/nutraceutical formulation in an amount of from about 0.01% to about 30% by weight, e.g., from about 0.01% to about 0.1%, from about 0.1% to about 0.5%, from about 0.5% to about 1.0%, from about 1.0% to about 2.0%, from about 2.0% to about 5%, from about 5% to about 7%, from about 7% to about 10%, from about 10% to about 15%, from about 15% to about 20%, from about 20% to about 25%, or from about 25% to about 30% by weight.

In some embodiments, the bacteria present in the food product/nutraceutical formulation are all the same species. In other embodiments, the bacteria in the food product/nutraceutical formulation comprise bacteria of two or more different species.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a solid or semi-solid food product, e.g., a bar, tablet, solid candy, lozenge, etc., the food product generally contains, by volume, less than about 15% water, e.g., from about 2% to about 5%, from about 5% to about 7%, from about 7% to about 10%, from about 10% to about 12%, or from about 12% to about 15% water. In some embodiments, the food product is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Other components discussed above, if present, are present in amounts ranging from about 0.001% to about 5% by weight of the composition.

In particular embodiments of interest, a subject lethally irradiated bacterial composition is formulated for oral delivery in a form that provides for increased transit time in the gastrointestinal tract. Such oral dosages forms include lozenges, hard candies, tablets, etc. that are kept in the mouth and allowed to dissolve in the mouth of the individual. For example, in some embodiments, a unit dosage form is a lozenge comprising an effective amount of lethally irradiated bacteria, and one or more of a flavoring, a sweetener, and a food coloring.

Package Comprising a Subject Food Product/Nutraceutical

The present invention further provides a package comprising a subject food product; comprising a subject nutraceutical; comprising a subject bacterial composition; or comprising a subject immunogenic composition. In some embodiments, a subject package comprises a single dosage form of a subject food product or bacterial composition. In other embodiments, a subject package a subject package comprising multiple (e.g., 2, 3, 4, 5, 6, 7, 8, 9, 10 or more) dosage forms of a subject food product or bacterial composition.

As one non-limiting example, a subject food product can be packaged in such a way that multiple doses are contained in a single package, optionally where individual, unit dosage forms (e.g., tablets, lozenges, etc.) are separated in individual compartments in a single package. The dosage forms can be in a variety of forms, e.g., tablets or lozenges that are palatable (e.g., flavored so as to be palatable, such as with fruit flavorings, sugars, and the like, as discussed above).

A subject package in some embodiments will further include instructions for use, including e.g., dosage amounts and dosage frequencies. Instructions are in some embodiments printed directly on the package. In other embodiments, instructions are printed material provided as a package insert. Instructions can also be provided in other media, e.g., electronically in digital or analog form, e.g., on an audio cassette, an audio tape, a compact disc, a digital versatile disk, and the like.

Formulations Suitable for Delivery by Inhalation

A subject lethally irradiated bacterial composition may be administered to an individual by means of a pharmaceutical delivery system for the inhalation route (oral, intratracheal, intranasal). Thus, a subject lethally irradiated bacterial composition may be formulated in a form suitable for administration by inhalation. The pharmaceutical delivery system is one that is suitable for respiratory therapy by topical administration of a subject bacterial composition to mucosal linings of the bronchi. This invention can utilize a system that depends on the power of a compressed gas to expel the bacteria from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains the bacteria, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

A subject lethally irradiated bacterial composition can also be delivered to the respiratory tract with a nebulizer, which is an instrument that generates very fine liquid particles of substantially uniform size in a gas. In many embodiments, a liquid containing a subject bacterial composition is dispersed as droplets. The small droplets can be carried by a current of air through an outlet tube of the nebulizer. The resulting mist penetrates into the respiratory tract of the patient.

A powder composition containing a subject bacterial composition, with or without a lubricant, carrier, or propellant, can be administered to a mammal. This embodiment of the invention can be carried out with a conventional device for administering a powder pharmaceutical composition by inhalation. For example, a powder mixture of a subject bacterial composition and a suitable powder base such as lactose or starch may be presented in unit dosage form in for example capsular or cartridges, e.g. gelatin, or blister packs, from which the powder may be administered with the aid of an inhaler.

There are several different types of inhalation methodologies which can be employed in connection with the present invention. A subject bacterial composition can be formulated in basically three different types of formulations for inhalation. First, a subject bacterial composition can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, a subject bacterial composition can be formulated in aqueous or ethanolic solutions and delivered by conventional nebulizers. In some embodiments, such solution formulations are aerosolized using devices and systems such as disclosed within U.S. Pat. No. 5,497,763; 5,544,646; 5,718,222; and 5,660,166.

Furthermore, a subject bacterial composition can be formulated into dry powder formulations. Such formulations can be administered by simply inhaling the dry powder formulation after creating an aerosol mist of the powder. Technology for carrying such out is described within U.S. Pat. No. 5,775,320 and U.S. Pat. No. 5,740,794.

Formulations suitable for intranasal administration include nasal sprays, nasal drops, aerosol formulations; and the like.

Dosages

A subject immunogenic composition is administered in an “effective amount” that is, an amount of a subject bacterial composition that is effective in a selected route of administration to elicit or induce an immune response. In some embodiments, an immune response is elicited to antigens produced by a pathogenic microorganism. In some embodiments, the amount of a subject composition is effective to facilitate protection of the host against infection, and/or to reduce a symptom associated with infection, by a pathogenic organism.

Dosages contain an amount of lethally irradiated bacteria in the range of from about 10 bacteria per dose to about 10⁷ bacteria per dose, e.g., from about 10 bacteria per dose to about 10² bacteria per dose, from about 10² bacteria per dose to about 5×10² bacteria per dose, from about 5×10² bacteria per dose to about 10³ bacteria per dose, from about 10³ bacteria per dose to about 5×10³ bacteria per dose, from about 5×10³ bacteria per dose to about 10⁴ bacteria per dose, from about 10⁴ bacteria per dose to about 5×10⁴ bacteria per dose, from about 5×10⁴ bacteria per dose to about 10⁶ bacteria per dose, from about 10⁶ bacteria per dose to about 5×10⁶ bacteria per dose, or from about 5×10⁶ bacteria per dose to about 10⁷ bacteria per dose.

Routes of Administration

In general, a subject irradiated bacterial composition/immunogenic composition is administered via a mucosal route of administration. In some embodiments, a subject bacterial composition is administered orally. In some embodiments, a subject bacterial composition is administered nasally or intranasally. Administration includes self-administration, e.g., where a subject bacterial composition is a food product, the individual ingests the food product orally. In other embodiments, a subject bacterial composition is administered by inhalation, e.g., intranasal inhalation or oral inhalation. In these embodiments, an individual or a medical personnel introduces a subject bacterial composition into the respiratory tract intranasally or orally by means of a aerosol delivery device, a metered dose inhaler, and the like. In other embodiments, a subject bacterial composition is administered intrarectally. In other embodiments, a subject bacterial composition is administered intravaginally.

Methods of Inducing an Immune Response

The instant invention provides methods of inducing an immune response in an individual to an antigen, the methods generally involving administering to an individual in need thereof an effective amount of a subject bacterial composition. In some embodiments, a subject method provides for inducing an immune response to a pathogenic microorganism. In other embodiments, a subject method provides for inducing a Th1-type immune response to an allergen. In other embodiments, a subject method provides for inducing an immune response to a tumor-associated antigen.

A subject immunogenic composition is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, multiple doses are administered. When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose.

Inducing an Immune Response to a Microbial Pathogen

Using the methods and compositions described herein in connection with the subject invention, an immune response, e.g., an immunoprotective response, against microbial pathogen can be induced in any mammalian subject, human or non-human, susceptible to infection by a microbial pathogen.

The present invention further provides methods for preventing or treating an infectious disease in an individual, comprising administering a subject formulation comprising lethally irradiated bacteria, in an amount effective to prevent or treat the disease. The methods are particularly useful for preventing or treating infectious diseases caused by intracellular pathogens, such as viruses, intracellular bacteria, fungi and parasites (e.g. protozoa, helminths, etc.). In addition, opportunistic infections can be treated using the methods of the invention.

“Preventing an infectious disease,” as used herein, refers to reducing the likelihood that an individual, upon infection by a pathogenic microorganism, will exhibit the usual symptoms of a disease caused by a pathogenic microorganism.

“Treating an infectious disease,” as used herein, encompasses reducing the number of pathogenic agents in the individual (e.g., reducing viral load, reducing bacterial load, reducing the number of protozoa, reducing the number of helminths) and/or reducing a parameter associated with the infectious disease, including, but not limited to, reduction of a level of a product produced by the infectious agent (e.g., a toxin, an antigen, and the like); and reducing an undesired physiological response to the infectious agent (e.g., fever, tissue edema, and the like).

The methods are effective to treat an infectious disease by at least about 5%, at least about 10%, at least about 20%, at least about 25%, at least about 50%, at least about 75%, at least about 85%, or at least about 90%, up to total eradication of the infecting pathogen and/or an associated parameter, when compared to a suitable control. Thus, in these embodiments, an “effective amount” of a subject immunogenic composition is an amount sufficient to treat an infectious disease, e.g., to reduce the number of pathogens and/or reduce a parameter associated with the presence of a pathogen, by at least about 5%, at least about 10%, at least about 20%, at least about 25%, at least about 50%, at least about 75%, at least about 85%, or at least about 90%, up to total eradication of the infectious disease, when compared to a suitable control. In an experimental animal system, a suitable control may be a genetically identical animal not treated with the subject composition. In non-experimental systems, a suitable control may be the infectious disease present before administering the subject composition. Other suitable controls may be a placebo control.

Whether an infectious disease has been treated can be determined in any of a number of ways, including but not limited to, measuring the number of infectious agents in the individual being treated, using methods standard in the art; measuring a parameter caused by the presence of the pathogen in the individual, e.g., measuring the levels of a toxin produced by the pathogen; measuring body temperature; measuring the level of any product produced by the pathogen; measuring or assessing any undesired physiological parameter associated with the presence of an infectious agent in an individual. Measuring the number of infectious agents can be accomplished by any conventional assay, such as those typically used in clinical laboratories, for evaluating numbers of pathogens present in a biological sample obtained from an individual. Such methods have been amply described in the literature, including, e.g., Medical Microbiology 3rd Ed., (1998) P. R. Murray et al., eds. Mosby-Year Book, Inc. A level of a product, including a toxin, produced by a pathogen can be measured using conventional immunological assays, using antibody which detects the product, including, but not limited to ELISA, RIA, protein blot assays, and the like. Other suitable assays include in vivo assays for the presence and/or level of bacterial toxins.

Whether an immune response has been elicited to a pathogenic organism can be determined (quantitatively, e.g., by measuring a parameter, or qualitatively, e.g., by assessing the severity of a symptom, or by detecting the presence of a particular parameter) using known methods. Methods of measuring an immune response are well known in the art and include immunological assays (ELISA, RIA, etc.) for detecting and/or measuring antibody specific to a given pathogenic organism; and in vitro assays to measure a cellular immune response (e.g., a CTL assay using labeled, inactivated cells expressing the epitope on their cell surface with major histocompatibility (MHC) Class I molecules).

A biological sample obtained from the individual is used to test for the presence and/or quantity of antigen-specific antibody (e.g., serum IgG, mucosal IgA, etc.); and/or antigen-specific CD4 response and/or CTL response. Suitable biological samples include, but are not limited to, serum; vaginal samples (e.g., fluids, cells); rectal samples (e.g., fluids, cells, etc.); blood; plasma; urine; lung lavage samples; sputum; and the like.

Whether a mucosal immune response is elicited can be determined using any known method, including, e.g., measuring secretory IgA, specific for an epitope(s) associated with the pathogenic organism, produced in a mucosal tissue.

Whether an immune response is effective to facilitate protection of the host against infection, or reduce symptoms associated with infection, by a pathogenic microorganism can be readily determined by those skilled in the art using standard assays, e.g., determining the number of pathogenic organisms in a host (e.g., measuring bacteria in a biological sample; measuring the number of helminths in a biological sample; measuring the number of a pathogen's eggs in a biological sample; and the like); measuring a symptom caused by the presence of the pathogenic organism in the host (e.g., elevated body temperature; and the like).

Treating an Allergic Disorder

In some embodiments, where a subject composition comprises lethally irradiated bacteria and an allergen, the invention provides methods of treating an allergy, e.g., reducing a Th2 immune response to the allergen. The methods involve administering to an individual who is sensitized to an allergen an effective amount of a subject composition comprising lethally irradiated bacteria and an allergen. In some embodiments, the methods further comprise administering to the individual at least one additional therapeutic agent for the treatment of an allergic disorder. In some embodiments, the allergic disorder is allergic asthma. In some embodiments, the allergic disorder is an allergic reaction to a plant allergen, a food allergen, an animal allergen, or a drug allergen. In some embodiments, the allergic disorder is selected from atopic dermatitis, a food allergy, allergic asthma, allergic gastroenteritis, and allergic rhinitis.

A subject method of treating an allergic disorder generally involves administering a subject formulation to an individual who is sensitized to an antigen (e.g., an allergen). A subject formulation is administered in an amount effective to treat the allergic disorder, e.g., to reduce production of IgE specific for the antigen (e.g., the allergen); to reduce the severity of a symptom of the allergic disorder; to reduce the amount of a conventional therapeutic agent that is required to treat the disorder; to reduce the frequency and/or severity of an allergic reaction to the allergen; and the like. Thus, e.g., an effective amount of a subject formulation is an amount that reduces the severity of a symptom and/or reduces a measurable parameter associated with the allergic disorder by at least about 10%, at least about 20%, at least about 25%, at least about 30%, at least about 40%, at least about 50%, at least about 60%, at least about 70%, at least about 80%, or at least about 90% or more, when compared with the symptom (e.g., the severity of the symptom), or when compared with the measurable parameter associated with the allergic disorder, in the absence of treatment with a subject formulation.

In some embodiments, an effective amount of a subject formulation reduces the level of serum IgE in an individual by at least about 10%, at least about 20%, at least about 25%, at least about 30%, at least about 40%, at least about 50%, at least about 60%, at least about 70%, at least about 80%, or at least about 90% or more, when compared with the level of serum IgE in the absence of treatment with a subject formulation. In some embodiments, an effective amount of a subject formulation reduces the severity of symptoms (e.g., reduces the frequency of coughing, sneezing, wheezing, etc.) by at least about 10%, at least about 20%, at least about 25%, at least about 30%, at least about 40%, at least about 50%, at least about 60%, at least about 70%, at least about 80%, or at least about 90% or more, when compared with the frequency of coughing, sneezing, wheezing, etc. in the absence of treatment with a subject formulation.

The efficacy of a subject method in treating an allergic disorder can be monitored according to clinical protocols well known in the art for monitoring the treatment of allergic disorders. For example, such clinical parameters as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5) can be monitored for determining efficacy. Indicators of efficacy of the treatment can include a reduction in severity and/or absence of symptoms, an increase in the number of symptom-free days per time period (e.g., per month) and/or a reduction in the need for conventional medications such as decongestants, anti-histamines, mast cell stabilizers and corticosteroids.

If the treatment of this invention is carried out in conjunction with immunotherapy, efficacy can be evaluated by observing an increase in tolerated dose of a given allergen(s). These parameters can be monitored weekly or monthly, as well as at greater time intervals (e.g., every 3-6 months). In a particular example, clinical parameters that can be monitored for asthma can include the number and severity of attacks as determined by symptoms of wheezing, shortness of breath and coughing. The measurement of airway resistance by the use of respiratory spirometry, the extent of disability and the dependence on immunosuppressive medications or bronchodilators can also be determined.

The efficacy of treatment for preventing an allergic disorder in a subject not known to have an allergic disorder, but known to be at risk of developing an allergic disorder, can be determined by evaluating clinical parameters such as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5), over time following administration of the nucleic acid or fusion protein of this invention. This time interval can be very short (i.e, minutes/hours) or very long (i.e., years/decades). The determination of who would be at risk for the development of an allergic disorder would be made based on current knowledge of the known risk factors for a particular allergic disorder as would be familiar to clinicians and researchers in this field, such as a particularly strong family history of an allergic disorder or exposure to or acquisition of factors or conditions (i.e., environmental factors or conditions) which are likely to lead to development of an allergic disorder.

In some embodiments, the methods further comprise administering to the individual at least one additional therapeutic agent for the treatment of an allergic disorder. Suitable therapeutic agents for the treatment of allergies which can be administered in a combination therapy with a subject composition for the treatment of allergic disorders include, but are not limited to, antihistamines such as loratadine (Claritin®), fexofenadine (Allegra®), terfenadine; astemizole, cetirizine, hydroxyzine, diphenhydramine; leukotriene synthesis inhibitors zileutron (Zyflo®); leukotriene receptor antagonists such as zafirlukast (Accolate®), and montelukast; β-adrenergic agonists such as epinephrine, isoproterenol, isoetharine, metaproterenol, albuterol, terbutaline, bitolterol, pirbuterol, and salmeterol; proinflammatory cytokine antagonists; proinflammatory cytokine receptor antagonists; anti-CD23; anti-IgE; anticholinergics such as atropine and ipratropium bromide; immunomodulating drugs; glucocorticosteroids; steroid chemical derivatives; anti-cyclooxygenase agents; anti-cholinergic agents; methylxanthines, cromones; anti-CD4 reagents; anti-IL-5 reagents; anti-thromboxane reagents; anti-serotonin reagents; ketotiphen; cytoxin; cyclosporin; methotrexate; macrolide antibiotics; heparin; and low molecular weight heparin.

Inducing an Immune Response to Cancer Cells

The present invention further provides a method of inducing an immune response, particularly a CTL response, to a cancer cell in an individual, the method generally involving administering to an individual having a cancer an effective amount of a subject composition or formulation comprising lethally irradiated bacteria an a tumor-associated antigen.

The methods are effective to reduce a tumor load by at least about 5%, at least about 10%, at least about 20%, at least about 25%, at least about 50%, at least about 75%, at least about 85%, or at least about 90%, up to total eradication of the tumor, when compared to a suitable control. Thus, in these embodiments, an “effective amount” of a subject composition comprising lethally irradiated bacteria and a tumor antigen is an amount sufficient to reduce a tumor load by at least about 5%, at least about 10%, at least about 20%, at least about 25%, at least about 50%, at least about 75%, at least about 85%, or at least about 90%, up to total eradication of the tumor, when compared to a suitable control. In an experimental animal system, a suitable control may be a genetically identical animal not treated with the subject composition. In non-experimental systems, a suitable control may be the tumor load present before administering the subject composition. Other suitable controls may be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen); computed tomography scanning, magnetic resonance imaging, and/or x-ray imaging of the tumor to estimate and/or monitor tumor size; measuring the amount of tumor-associated antigen in a biological sample, e.g., blood; and the like.

Subjects Suitable for Treatment

Subjects suitable for treatment with the methods of the invention include an individual who has been infected with a pathogenic microorganism; an individual who is susceptible to infection by a pathogenic microorganism, but who has not yet been infected; an individual who has or who is at risk of having an allergic disorder; and an individual who has a tumor. In many embodiments of interest, the subject is a human. In other embodiments of interest, the subject is a non-human mammal, e.g., a livestock animal (e.g., an ungulate such as a horse, sheep, goat, pig, or cow); a canine (e.g., a dog); a feline (e.g., a cat); or other non-human mammal.

Immunization Methods

Subjects suitable for treatment with a subject method of inducing an immune response to a microbial pathogen, and methods of treating or preventing an infection with a microbial pathogen, include individuals who have been infected with a pathogenic microorganism; individuals who are susceptible to infection by a pathogenic microorganism, but who have not yet been infected; and individuals who are at risk of becoming infected with a pathogenic microorganism, but who have not yet been infected. Suitable subjects include infants, children, adolescents, and adults.

Subjects suitable for treatment with a subject method of inducing an immune response to a microbial pathogen, and methods of treating or preventing an infection with a microbial pathogen, include pediatric target population, e.g., individuals between about 1 year of age and about 17 years of age, including infants (e.g., from about 1 month old to about 1 year old); children (e.g., from about 1 year old to about 12 years old); and adolescents (e.g., from about 13 years old to about 17 years old).

Subjects suitable for treatment with a subject method of inducing an immune response to a microbial pathogen, and methods of treating or preventing an infection with a microbial pathogen, include CD4⁺-deficient individuals, e.g., individuals who have lower than normal numbers of functional CD4⁺ T lymphocytes. As used herein, the term “normal individual” refers to an individual having CD4⁺ T lymphocyte levels and function(s) within the normal range in the population, for humans, typically 600 to 1500 CD4⁺ T lymphocytes per mm³ blood. CD4⁺-deficient individuals who have an acquired immunodeficiency, or a primary immunodeficiency. An acquired immunodeficiency may be a temporary CD4⁺ deficiency, such as one caused by radiation therapy, or chemotherapy, as described below.

Also suitable for treatment with the methods of the invention are individuals with healthy, intact immune systems, but who are at risk for becoming CD4⁺ deficient (“at-risk” individuals). At-risk individuals include, but are not limited to, individuals who have a greater likelihood than the general population of becoming CD4⁺ deficient. Individuals at risk for becoming CD4⁺ deficient include, but are not limited to, individuals at risk for HIV infection due to sexual activity with HIV-infected individuals; intravenous drug users; individuals who may have been exposed to HIV-infected blood, blood products, or other HIV-contaminated body fluids; babies who are being nursed by HIV-infected mothers;

Subjects suitable for treatment with a subject method for treating cancer include individuals who have been diagnosed with cancer; individuals who were previously treated for cancer, e.g., by chemotherapy or radiotherapy, and who are being monitored for recurrence of the cancer for which they were previously treated; and individuals who have undergone bone marrow transplantation or any other organ transplantation.

Subjects suitable for treatment with the formulations and methods of the instant invention include any individual who has been diagnosed as having an allergy. Subjects amenable to treatment using the methods and agents described herein include individuals who are known to have allergic hypersensitivity to one or more allergens. Subjects amenable to treatment include those who have any of the above-mentioned allergic disorders. Also amenable to treatment are subjects that are at risk of having an allergic reaction to one or more allergens. Also suitable are individuals who failed treatment with one or more standard therapies for treating an allergic disorder.

Subjects suitable for treatment include individuals living in industrialized nations; individuals living developing countries; individuals living in rural areas; individuals living in relatively isolated areas; and the like.

The target population for a subject immunogenic composition will vary, depending on the microbial pathogen. Use of a subject immunogenic composition is not contraindicated in infants, children, or immunocompromised or immunosuppressed adults.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec., second(s); min, minute(s); h or hr, hour(s); sc, subcutaneous, subcutaneously; ip, intraperitoneally; im, intramuscular; and the like.

### Example 1

### Comparative Efficacy of γ-Irradiated and Heat-Killed Listeria monocytogenes Vaccines

In general, killed bacterial vaccines have not been protective against intra-cellular bacterial pathogens such as M. tuberculosis and Listeria monocytogenes, whereas live vaccines are protective. The possibility that the usual methods of bacterial inactivation, such as heating or formalin fixation, may have been the problem, was considered, based on early experiments showing that irradiated, but not boiled, Lactobacilli retained TLR9 stimulating capability. Irradiated bacteria were tested as a vaccine in two standard mouse models of infection. Listeria monocytogenes, strain 1043 s (L.m.), was used. The bacteria were grown in trypticase soy broth (TSB) overnight, washed three times and resuspended in saline. The suspension was divided and some were γ-irradiated using a cesium (Cs) source for 3 hours; and the remainder were heat killed (HK) using heat treatment at 70.5° C. for 1 hour. It was confirmed that 100% of the bacteria were dead in both groups by sub-culturing the equivalent of 10⁸ bacteria in Trypticase Soy Broth (TSB) for two days; after two days of culturing in these conditions, there was no detectable growth. C57BL/6 (B6) mice were immunized with the irradiated or heat killed (HK) bacteria by two subcutaneous injections of 0.1 ml of bacteria one week apart. The volume administered contained the equivalent of 10¹⁰ colony forming units (CFU) to maximize the chance that HK L.m. would work. A positive control was 10⁴ CFU of live bacteria i.p., given at the same time as the first immunization. Immunized and control B6 mice were challenged three weeks after the second immunization with 6×10⁶ CFU i.p (10×LD₁₀₀). Three days after challenge, the mice were sacrificed and their spleens removed for quantitative cultures. As shown in Table 1, the live vaccine was most effective, reducing the bacterial counts by 5 logs. However, mice immunized with the irradiated bacteria had only 1% of the number of bacteria present in the controls (P=<0..01). In contrast, the HK vaccine was ineffective, with a statistically insignificant reduction of about 50%.

TABLE 1
Efficacy of a γ-irradiated Listeria monocytogenes vaccine:
* 3 days after infection, # Geometric mean; 8 mice/group

| | CFU/spleen* |
|---|---|
| Immunization | (log₁₀) |
| None | 7.3# |
| HK L.m. | 6.9 |
| Irrad. L.m. | 5.4 |
| Live L.m. | 2.1 |

### Example 2

### Comparative Efficacy of γ-Irradiated and Heat-Killed Salmonella dublin Vaccines

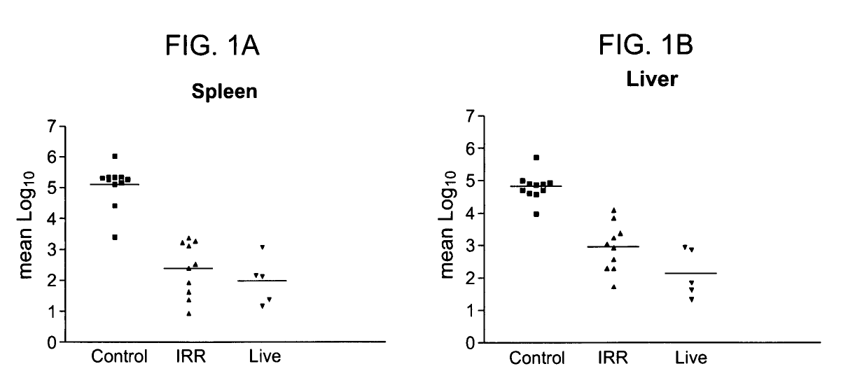

FIGS. 1A and 1B. To determine whether the effect of y-irradiation extended to gram-negative bacteria, infection of mice with Salmonella dublin was studied. As the live vaccine strain, LD842, an isogenic strain from which the virulence plasmid has been removed, was used. LD842 was compared to LD842 that was y-irradiated. In order to kill 100% of the Salmonella, the duration of the irradiation was increased to 18 hours (1.2 mR). Again, Balb/c.D2 (Nramp1 congenic) mice were immunized with two injections of γ-irradiated bacteria subcutaneously one week apart. These mice are genetically resistant to Salmonella infections because they do not have the mutant Nramp1 gene. (Normal people and non-inbred mice have wild-type Nrmp1 genes.) Mice were challenged with 1.2×10³ live S. dublin two weeks after the last immunization. Mice were sacrificed 5 days after challenge; and the number of bacteria in spleen and livers of the infected mice was counted. Control mice were unimmunized; and were challenged with the live Salmonella dublin. As shown in FIGS. 1A and 1B, there was nearly a three log reduction of bacteria in the livers and spleens of mice that received γ-irradiated LD842 (“IRR”). This was nearly as effective as the live vaccine (“Live”).

### Example 3

### Comparative Efficacy of γ-Irradiated and Heat-Killed Listeria monocytogenes Vaccines

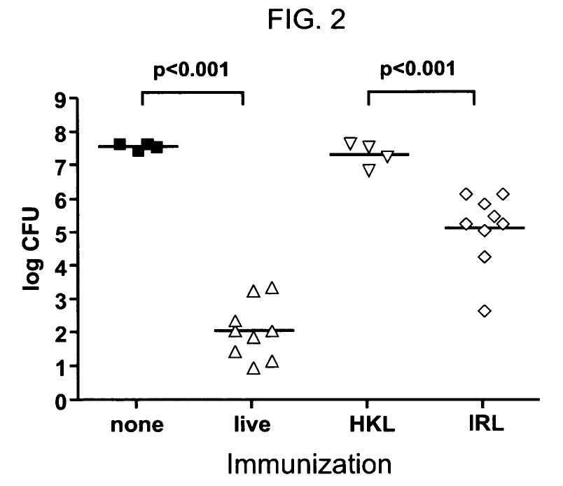

FIG. 2. Listeria monocytogenes (LM) 10403 S were cultured overnight at 37° C. in tryptic soy broth (TSB) and then resuspended in saline. An aliquot of this suspension was plated on tryptic soy agar (TSA) to determine the concentration of bacteria. Remaining aliquots of the bacterial suspension were then γ-irradiated, using a JL Sheperd Mark I Model 30 irradiator, with 600 Krad over six hours to prepare irradiated LM (IRL), or heated to 70° C. for one hour-to prepare heat-killed LM (HKL). The sterility of the IRL and HKL preparations was verified by lack of growth after incubating the equivalent of 10⁹ colony forming units (CFU) in TSB and TSA for 48 hours at 37° C. C57B1/6 mice were then left untreated (“none”); immunized intraperitoneally with live LM (10⁴ bacteria/mouse) on day 0 (“live”); or immunized subcutaneously at the tail base with HKL or IRL (10⁹ bacteria/mouse) on day 0 and day 7 (“HKL” and “IRL”). The mice were then challenged intraperitoneally on day 28 with 10⁶ CFU of live LM. After sacrifice of the mice on day 31, the ground spleen of each mouse was serially diluted, plated on TSA, and CFU/spleen determined. The result are shown in FIG. 2. Each point on the graph shown in FIG. 2 represents the CFU/spleen for a single mouse, and the mean CFU for each group is shown by the line. The data depicted in FIG. 2 show that immunization with IRL elicits significant protective immunity that is not elicited by HKL.

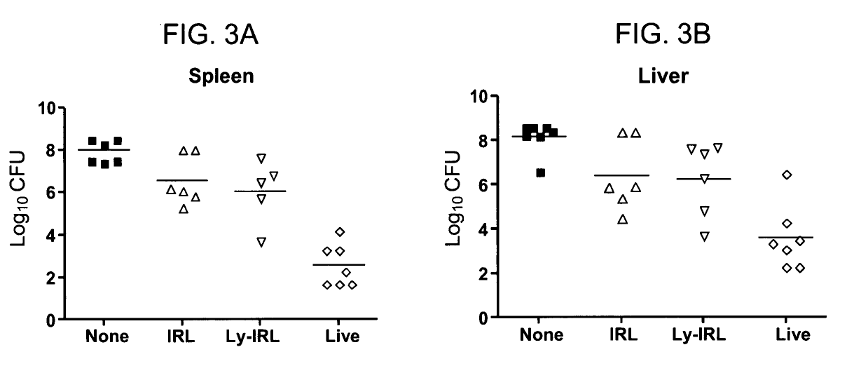

FIGS. 3A and 3B. IRL were prepared as described as above and aliquoted into sterile microfuge tubes and placed on a speed-vac until dry. Sterility of a representative lyophilized sample was verified by lack of growth in TSB and TSA as described above. C57B1/6 mice were left unimmunized (“none”); immunized subcutaneously with freshly irradiated LM (IRL, 109 bacteria/mouse) on day 0 and day 7 (“IRL”); immunized subcutaneously with lyophilized IRL (Ly-IRL, 10⁹ bacteria/mouse) on day 0 and day 7 (“Ly-IRL”); or immunized intraperitoneally with live LM on day 0 (“Live”). The mice were then challenged on day 56 as described above, and CFU per spleen and CFU per liver were determined on day 59, as described above for FIG. 2. The results are shown in FIGS. 3A and 3B, where the CFU per spleen are shown in FIG. 3A and the CFU per liver are shown in FIG. 3B. The data depicted in FIGS. 3A and 3B show that lyophilized irradiated LM induce protective immunity similarly to freshly irradiated LM at 2 months post-immunization. Since lyophilization provides long-term stability of samples, this method will allow irradiated vaccine preparations to be more conveniently and practically stored over long time periods.

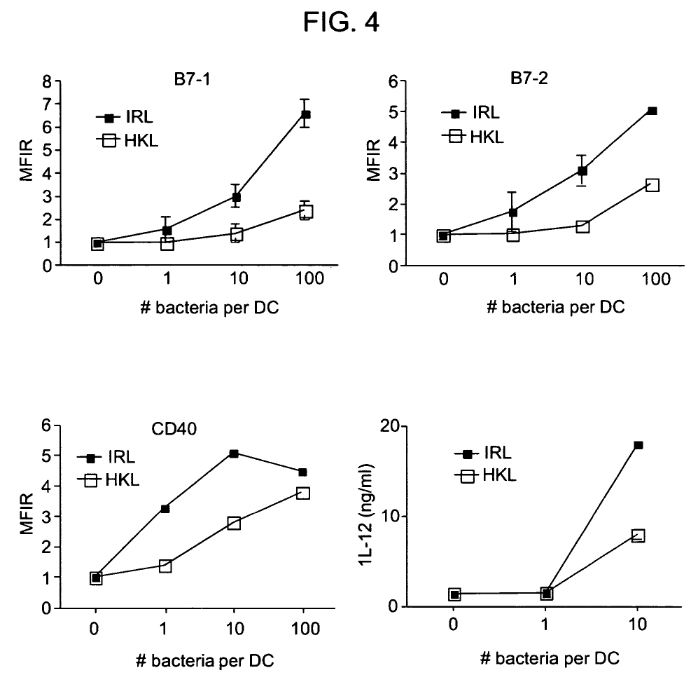

FIG. 4. Titrated numbers of irradiated (IRL) or heat-killed (HKL) Listeria were incubated with dendritic cells (DC) overnight. Cells were assessed by flow cytometry for B7-1, B7-2, and CD40 induction, expressed as mean fluorescence intensity ratio (MFIR) that was calculated as [mean fluorescence of treated cells]/[mean fluorescence of untreated cells]. IL-12 levels in the supernatant were determined by enzyme linked immunosorbent assay (ELISA). These results show that irradiated bacteria retain adjuvant properties that are lost with heat-killing. This retention in adjuvant properties contributes to their improved ability to elicit protective immunity.

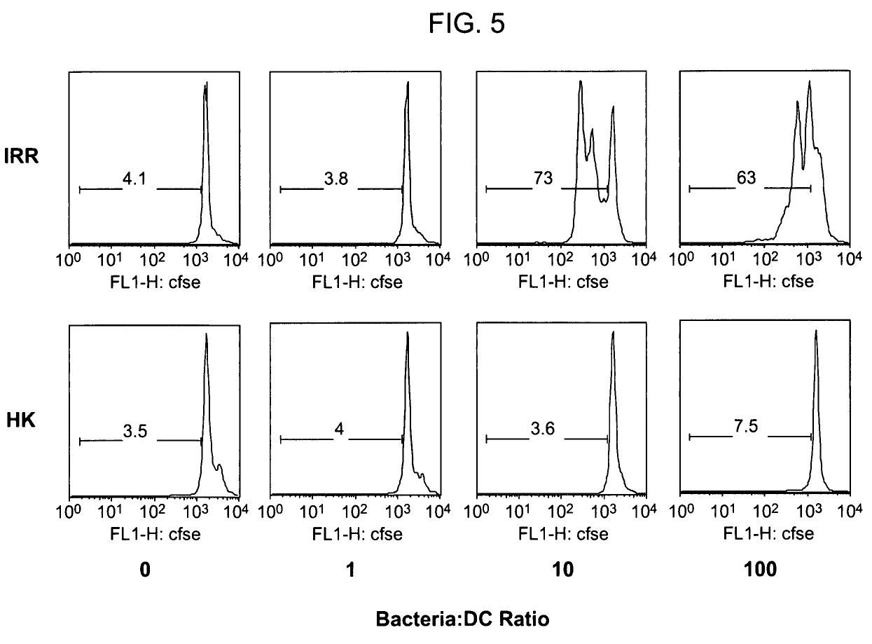

FIG. 5. Titrated numbers of irradiated (IRR) or heat-killed (HK) ovalbumin-expressing Listeria (LM-OVA) were incubated with DC overnight. Listeria monocytogenes was genetically modified to produce ovalbumin (OVA). Genetically modified, OVA-producing Listeria monocytogenes cells, either IRR or HK, were incubated with DC overnight. The DC were then washed and incubated for 3 days with CarboxyFluorescein Diacetate Succinimidyl Ester (CFSE)-labeled, OVA-specific CD8⁺ T cells from OT-I mice. Histograms show CFSE intensity of CD8⁺ cells as determined by flow cytometry; percentage of cells undergoing at least one division is noted within each histogram. These results show that irradiated bacteria retain antigenic properties that are lost with heat-killing, allowing them to better activate CD8⁺ T cells.

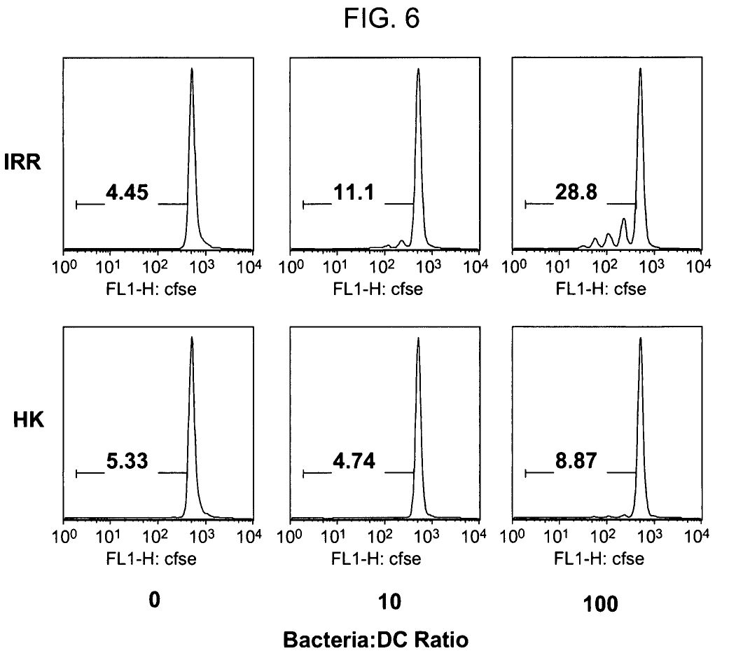

FIG. 6. DC were incubated with IRR or HK LM-OVA, and their ability to activate CFSE-labeled, OVA-specific CD4⁺ T cells from OT-II mice was determined by flow cytometry as described above for FIG. 5. These results show that irradiated bacteria retain antigenic properties, whereas such antigenic are lost with heat killing. Retention of antigenic properties allows irradiated bacteria to better activate CD4⁺ T cells.

Examples 1-3 show that that protection of mice against infection with two genetically unrelated facultative intra-cellular pathogens using γ-irradiated, killed bacteria as a vaccine was achieved.

### Example 4

### Multimerization of TLR Ligands

Experimental Design

Synthesis of multimeric TLR7-9 ligands: The ISS-ODN (e.g., 1018, sequence 5′-TGACTGTGAACGTTCGAGATGA-3′; SEQ ID NO:3) an activator of TLR9 that contains both murine and human CpG motifs was purchased from Trilink (San Diego, Calif.). Phosphorothioate [PS] and phosphodiester (PO) ODNs containing the TLR7 activators 7-thia-8-oxodeoxyguanosine (TOG) or 7-deazadeoxyguanosine (7DG) were prepared by standard DNA synthesis techniques using the phosphoramidite chemistry approach. Both PO and PS backbones were included. ODNs containing the TLR7-8 activator R-848 were prepared by coupling amino modified R-848 (see synthetic scheme below) to modified ODNs with an aldehyde at the 5′-end. The TLR7 and TLR8 ligands were incorporated into ODNs with and without TLR9 activating CpG motifs.

ODNs containing exclusively TOG or 7DG in place of guanine residues would not be expected to form G-quartets and multimers because the H-binding nitrogens in the 7-position have been replaced by sulfur and carbon, respectively. ODNs with incorporated R-848 may also not aggregate. However, a short guanine stretch at the 3′-end of on ODN induces aggregation after incubation with neomycin or tobramycin. Accordingly, all ODNs were prepared with and without guanine tails.

Assessment of TLR ligand multimerization: The ISS-like molecules described above were characterized by non-denaturing gel electrophoresis, size exclusion HPLC, and circular dichroism spectroscopy to detect multimers and secondary structures that form in protein free isotonic buffers. To measure multimerization due to protein binding, a [³-H]- or [³²-P]- labeled base was incorporated into the ISS-like molecules. After incubation with mouse or human sera, size exclusion HPLC and scintillation counting are carried out to measure the distribution of radioactivity in monomeric and aggregated fractions. The ability of the aminoglycoside antibiotics tobramycin and neomycin to facilitate the formation of stable TLR ligand multimers was determined. Various molar ratios of ISS-like molecules and antibiotic, ranging from 1:1 to 20:1 were incubated overnight in isotonic saline ph 7.4, and then analyzed by non-denaturing electrophoresis. In some cases, mixed multimers were generated containing TLR7, 8, and 9 ligands. The multimers were visualized by dye staining, by UV shadowing, and by autoradiography. The size distributions were confirmed by size exclusion HPLC. Aliquots of the ODN antibiotic suspensions were distributed at varying concentrations in complete medium, just prior to testing for immunostimulatory activity. The aggregate fraction eluted from the HPLC columns was collected, diluted in medium with or without serum, incubated at 37° C. for 1-24 hours, and then rechromatographed.

In vitro screening systems to assess activity and synergism of the TLR ligands in monomeric and multimeric forms: The primary screen for the potency of the ISS-like molecules is activation of BMDC cells. Thus the induction of CD40, CD80 and CD86 was evaluated (by fluorescence activated cell sorter (FACS) analysis) and the production of IL-12 and IFNα was assessed (by ELISA). For human PBMC related studies, buffy coats from anonymous donors are purchased from the San Diego Blood Bank. The PBMC are isolated isopycnic centrifugation, and dispersed in microwell cultures at a density of 0.5×10⁶ per ml in complete medium containing 10% human serum supplemented with the ISS-like molecules. After 24 hours, supernatants are harvested and assayed for IFNγ and IFNα by ELISA. Only those ISS-like molecular multimers that are active toward both murine and human cells are candidates for further in vivo testing.

Incorporation of R-848 in ISS-like molecule: The TLR7-8 ligands such as R-848 was incorporated into ODNs by preparing an appropriate amino-modified spacer derivative and reacting that with the 5′-aldehydo derivative of the ODN to yield a Schiff base intermediate that is finally reduced to provide the stable conjugate. The synthetic strategy was as follows:

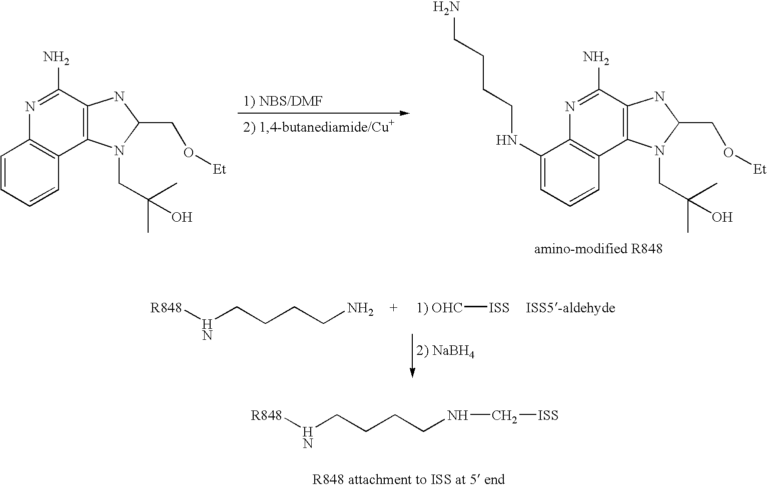

Commercially available R-848 was first modified by electrophilic bromination at the 6 position (or the 9 position) followed by copper catalyzed displacement of the bromine atom with 1,4-butanediamine. The resulting primary amine now serves as the linker/spacer for attachment to the aldehydo-ISS. Before conjugation reactions were undertaken, the amino-modified R848 was assayed to ensure that the modification was not abrogated all TLR-mediated immunostimulatory activity. As an alternative modification site, the 9 position of R848 can also be halogenated and may be preferable in the event that the 6 position modification is not suitable due to lack of immunoactivity.

Conjugation of TLR ligands to irradiated bacterial vaccine strain (LVS): Small molecules such as R-848 or ODNs such as ISS may be conjugated to bacteria by a number of methods that have been developed over the years for the covalent attachment of polysaccharides to proteins. See, e.g., Theilacker et al. (2003) Infect. Immun. 71:3875-3884; and Jin et al. (2003) Infect. Immun. 71:5115-5120. An important objective for the selected bioconjugation method is to avoid extensive modification/disruption of the bacterial antigenicity while providing sufficient linkages to the adjuvant molecule to promote good adjuvanticity. Conjugates are prepared based on chemistry developed by Solulink, Inc. (San Diego, Calif.; on the Internet at solulink.com). In this approach, amine containing ODNs or small molecules are modified to contain an aldehyde function as outlined in the following general scheme:

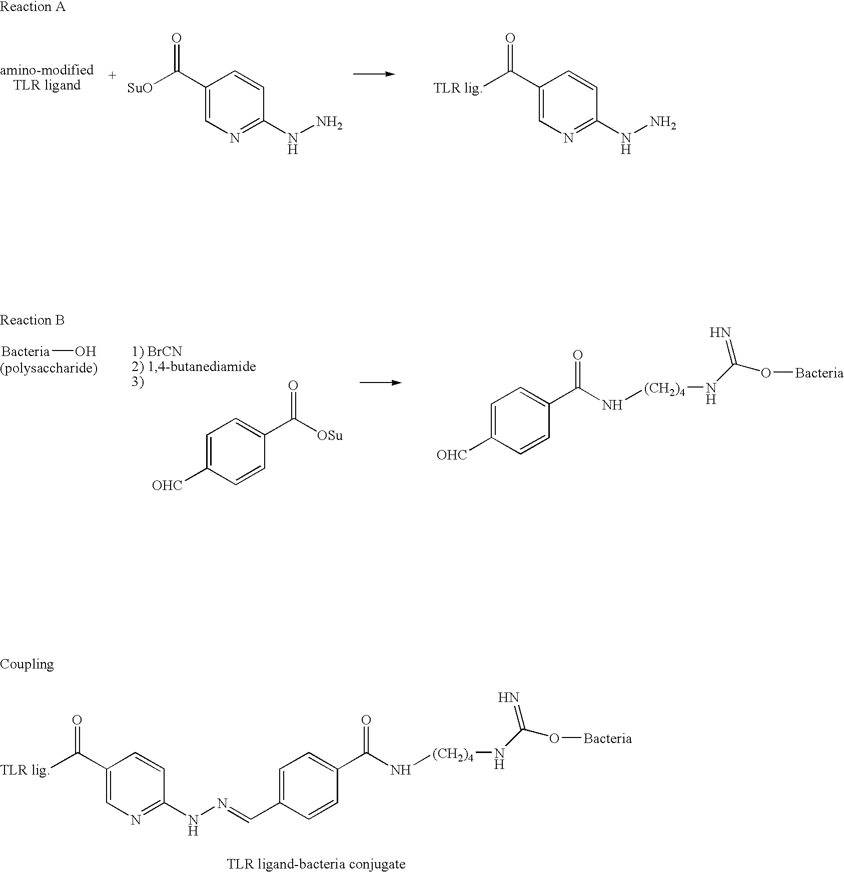

The amino-modified ISS and TOG are commercially prepared. The bacteria polysaccharide is also modified on several of the polysaccharide hydroxyls to contain a hydrazine moiety, also shown here (Reaction B). The aldehyde is then reacted under mild conditions with the hydrazine to produce a very stable hydrazone linkage. An advantage of the hydrazine/carbonyl coupling approach is that hydrazone formation produces a chromophore that absorbs at 360 nm, which is convenient for measuring the extent of conjugation. Moreover, the intermediates prepared for the coupling are stable in water and can be stored for months, if needed, without significant degradation.

Results

Two chemical strategies were devised to prepare and evaluate pre-formed multimeric TLR7-9 ligands. In the first set of experiments, immunostimulatory oligonucleotides (ISS-ODN; TLR9 ligands), or ODNs containing the TLR7 activators TOG and 7DG (47), and/or the TLR 7-8 activator R-848, were mixed with the aminoglycoside antibiotics tobramycin or neomycin, in order to form stable lattices of varying sizes. Tobramycin is a particularly attractive multimerization agent, since it is already approved for inhalation in humans. However, even neomycin should be safe, since the administered dosages are 1000 fold lower than TLR ligand plus the dosages used for anti-microbial therapy. The primary screen for the immunostimulating activities of the various TLR ligands is in vitro activation of mouse BM derived DC. To eliminate any adjuvants that are only active in mice human peripheral blood mononuclear cells (PBMC) is also analyzed.

Comparative ISS activities of oligodeoxynucleotide (ODN) multimers and monomers were analyzed. The multimeric and monomeric forms of various ODNs were collected from HPLC eluates, and diluted to equivalent OD₂₆₀ concentrations in phosphate buffered saline (PBS). Aliquots of the separate fractions were then incubated with BMDM for 2 days. The cell media were harvested and IL-12p40/p70 levels were determined by ELISA. These results are elaborated upon in Example 5, below.

Induction of ODN multimerization by tobramycin and neomycin was analyzed. Phosphodiester 7G3dG4 and phosphothioate SEQ ID NO:01-based ODN were synthesized by Trilink (San Diego, Calif.), and purified using reversed-phase HPLC. The aminoglycosides tobramycin and neomycin were purchased from Sigma. The ODNs and aminoglycosides were dissolved in PBS and mixed at several different molar ratios and left at room temperature for 10 minutes. The mixtures were-loaded onto 1.5% TAE agarose gels for electrophoresis. Following electrophoresis, the ODNs were visualized by dye staining and laser densitometry. The percent residual monomeric ODN was calculated by scanning the monomeric ODN bands shown in the inset panels, where 100%=band intensity without aminoglycoside antibiotic. The antibiotic-induced aggregates were very high molecular weight, since they formed a fine cloudy suspension and did not move through the agarose gels.

The immunostimulatory activity of ODN/neomycin multimers was analyzed. The indicated concentrations of the ISS-ODN of SEQ ID NO:3 alone, or the pre-formed ODN SEQ ID NO:3/neomycin multimer (molar ratio 1:3) were incubated for 48 hours with purified BMDM (bone marrow-derived macrophages). IL-6 and IL-12 in the supernatants were then measured by ELISA, and CD80 expression was assessed by FACS (fluorescence activated cell sorting) analyses. The results indicated that the 1018/Neo multimer (SEQ ID NO:3/Neo multimer) induced the production of approximately 15 ng IL-6, while media alone or neomycin alone induced production of undetectable quantities of IL-6; and 1018 alone induced production of IL-6 at a level that was barely above background. The results further indicated that the 1018/Neo multimer (SEQ ID NO:3/Neo multimer) induced the production of approximately 75 ng/ml IL-12, while media alone or neomycin alone induced production of undetectable quantities of IL-12; and 1018 alone induced production IL-12 at about 12 ng/ml.

The ODN-like activators of TLR7-9 are conjugated directly to the γ-irradiated LVS, using variants of chemical strategies that have been successfully employed in the development of vaccines against bacterial polysaccharides. The effects of the conjugates upon antibody recognition and/or bacterial uptake by APCs are assessed. The ability of the different conjugates to induce cell mediated immunity after i.d. administration in mice is determined. Finally, the most potent vaccines are tested for adjuvant activity in mice vaccinated with γ-irradiated LVS and challenged with the live LVS.

### Example 5

### Multimerization of TLR9 Ligands

Materials and Methods

Oligodeoxynucleotides-Phosphodiester oligodeoxynucleotides (ODNs) from a random library with scrambled insets of 40 nucleotides were selected for their ability to penetrate cells by a repetitive selection procedure that involved (a) incubation with viable cells, (b) extensive and stringent washing to remove all external ODN binding, and (c) asymmetric polymerase chain reaction (PCR) amplification, as described previously. Wu et al. (2003) Hum. Gene. Ther 14:849-860. After 10 rounds of selection, the retained intracellular ODNs were amplified, cloned, and sequenced. Several 40-mer ODNs corresponding to the recovered sequences as well as a random ODN of the same length (random 40) were synthesized by Integrated DNA Technologies (IDT, Corvallis, Oreg.). Nucleotide sequences of exemplary ODNs are shown in Table 2. Sequences are given in the 5′ to 3′ direction. CpG dinucleotides are in boldface type. Murine ISS motifs (5-purine-purine-CG-pyrimidine-pyrimidine) are underlined

TABLE 2

| | | | |
|---|---|---|---|
| ODN | Sequence | CpG | ISS |

| | | | |
|---|---|---|---|
| R10-5 | CCAGCCACCTACTCCACCAGTGCCAGGACTGCT | 0 | 0 |
| | TGAGGGG; SEQ ID NO:4 | | |
| R10-9 | CTAACGTTTAACCAGGATCCCCCAAGTCCCTGC | 1 | 1 |
| | TAGTGGG; SEQ ID NO:5 | | |
| R10-32 | TGGGCGTTACCACTACAGGTCCAGATTTGTCTG | 2 | 1 |
| | TCCGGGG; SEQ ID NO:6 | | |
| R10-71 | GGGATCTACGGCTAAACATCTAACGCTCTTTGG | 2 | 1 |
| | CCCTGGG; SEQ ID NO:7 | | |
| R10-13 | CGCTCCCTTATATATCCGACGTGACTAATACTG | 3 | 1 |
| | TGGGGC; SEQ ID NO:8 | | |
| R10-34 | GCACATAAAACTTTACCCCGACGTGGAGGACGT | 3 | 1 |
| | TCTTGGC; SEQ ID NO:9 | | |
| R10-60 | CCAGTCGTACAGGAAACATGCGTTCTAGATGTT | 3 | 0 |
| | CGGGGC; SEQ ID NO:10 | | |
| D-R15-8 | CGCAGCGTATGGATTCAGGGTTGGATCGTGTAG | 3 | 0 |
| | GGGGGG; SEQ ID NO:11 | | |
| R10-11 | CGCGTGAAGAAAAGGAGCAGTCATAAACGCTAA | 4 | 1 |
| | TCGTGCC; SEQ ID NO:12 | | |
| R10-53 | TCTGCGGGGAAGAGCTACGTTACTAGTCGTGTG | 4 | 0 |
| | TCCGTG; SEQ ID NO:13 | | |
| R10-86 | GCGGCCATTCAGGAAACGTTAATGTCGATCTAC | 4 | 1 |
| | GTTGGC; SEQ ID NO:14 | | |
| Control | CCTGGCTGTTCCGAAACATATCCACAGTTGTTG | 1 | 0 |
| random 40 | GCCCAGG; SEQ ID NO:15 | | |
| 1018 | TGACTGTGAACGTTCGAGATG; | 2 | 1 |
| | SEQ ID NO:3 | | |

The prototype ISS-ODNs, 1018 and 1826, have been described (Roman et al. ((1997) Nat. Med. 3:849-854); Magone et al. ((2000) Eur. J. Immunol. 30:1841-1850); Broide et al. ((2001) J. Clin. Immunol. 21:175-182); and Ballas et al. ((2001) J. Immunol. 167:4878-4886)) and were synthesized with both phosphodiester and phosphorothioate backbones. Endotoxin contamination in ODNs was negligible as measured by a Limulus amebocyte lysate assay (BioWhittaker, Walkersville, Md.).

Uptake studies were performed using fluorochrome-labeled ODNs to confirm their abilities to penetrate cells, as described. Wu et al. (2003) Hum. Gene. Ther. 14:849-860. Briefly, viable cells were incubated in protein-free medium with 5′-Cy3-labeled ODNs at 0.5 μM for 2 h at 37° C. Following washes with 3% fetal bovine serum in RPMI 1640 medium (Irvine Scientific, Santa Ana, Calif.), these cells were analyzed by flow cytometry using a Becton Dickinson FAC-Scaliber. Data analysis was carried out using FlowJo 3.4 software (Tree Star, Inc., Stanford, Calif.).

Isolation of Bone Marrow-derived Mononuclear Cells—BALB/c and C57BL/6 mice were purchased from The Jackson Laboratory (Bar Harbor, ME). MyD88^{-/-} mice are described in Adachi et al. (1998) Immunity 9:143-150. The mice were bred and maintained under standard conditions in the University of California, San Diego Animal Facility that is accredited by the American Association for Accreditation of Laboratory Animal Care. All animal protocols received prior approval by the institutional review board.

Bone marrow harvested from the femurs and tibias of various strains of mice were plated in non-tissue culture-treated Petri dishes with Dulbecco's modified Eagle's medium high glucose medium supplemented with 10% fetal bovine serum, L-glutamine, penicillin/streptomycin, all from Invitrogen, and 30% L929 cell-conditioned medium.

Cells were grown at 37° C., 5% CO₂ for 7 days without replacing the medium. The bone marrow-derived mononuclear cells were harvested afterward by gentle scraping, counted, and re-plated in medium with different conditions described below.

ODN-stimulated Cytokine Release—For studies on cytokine production, 7-day-old bone marrow-derived mononuclear cells were seeded in 96-well plates at a density of 5×10⁴ cells/well and grown for another 3 days. These cells were then incubated with ODNs at a final concentration of 0.2, 0.5, or 1 μM for 48 h without further supplement. In the competitive receptor binding study, the highly active ODN R10-60 (0.5 μM) was premixed with various concentrations of the inactive ODN R10-9, R10-32, or R10-13 in serum-free medium, prior to addition to the cells. Culture supernatants were collected at the end of incubation and stored at −20° C. for later determination of IL-12p40/70 by sandwich enzyme-linked immunosorbent assay (BD Biosciences).

In Vitro Kinase Assays-For kinase assays, the enriched mononuclear cells were dispersed in 6-well plates at a density of 1-2×10⁶ cells/ml/well and allowed to settle overnight. ODNs were then added to the mononuclear cells at 1 μM and incubated for 0.5-2 h. The cells were quickly lysed in buffer A (20 mM Hepes, pH 7.9, 1 mM EDTA, 1 mM EGTA, 1% Nonidet P-40, 1 mM glycerophosphate, 2.5 mM sodium pyrophosphate, 1 mM sodium orthovanadate, 2 mg/ml aprotinin, 1 mM phenylmethylsulfonyl fluoride) with proteinase inhibitors and 1 mM dithiothreitol on ice and centrifuged at 12,000×g for 1 min. The aqueous phase containing cytoplasmic proteins was removed and saved. The nuclear pellet was lysed in buffer B (20 mM Hepes, pH 7.9, 1 mM EDTA, 1 mM EGTA, 0.4 M NaCl) and vortexed, and the nuclear supernatant was collected after centrifugation.

Specific kinases were immunoprecipitated from cytosolic proteins with either anti-IκB kinase-β or anti-June NH₂-terminal kinase I antibodies (Santa Cruz Biotechnology, Inc., Santa Cruz, Calif.) at 4° C. overnight. Afterward, the immune complexes were washed successively in buffer A containing 0.5 M NaCl followed by kinase buffer (25 mM Tris, pH 7.5, 10 nM MgCl₂, 2 mM EGTA, 1 mM dithiothreitol, 1 mM sodium orthovanadate). IκB kinase-β or Jun NH₂-terminal kinase 1 kinase assays were performed using the respective recombinant glutathione S-transferase fusion protein with IκBα or c-Jun as the respective substrates in the presence of 0.1 μCi of [γ-³²P]ATP at 37° C. for 30 min, as described. Lee et al. (2000) J. Leukocyte Biol. 68:909-915. The ³²P labeled products were separated by sodium dodecyl sulfate-polyacrylamide gel electrophoresis (SDS-PAGE) and visualized by autoradiography.

DNA Secondary Structure Prediction—To predict the presence of secondary structures, the DNA mfold program (Zuker (2003) Nucleic Acids Res. 31:3406-3415) (available on the World Wide Web at bioinfo.rpi.edu/applications/mfold/old/dna/) was employed. The various sequences were submitted as linear DNA and analyzed based on free energy using default program settings, assuming a temperature of 37° C., with ionic conditions of 150 mM Na⁺ and 0.5 mM Mg²⁺.

Analysis of ODN Multimerization by Size Exclusion HPLC and PAGE—A TSK-Gel G2000SWXL HPLC column with a 5-μm particle size (MAC-MOD Analytical, Montgomeryville, Pa.) was used to perform the size exclusion assay as previously described. Wu et al. (2003) Hum. Gene Ther. 14:849-860; and Suzuki et al. (1999) Eur. J. Biochem. 260:855-860. Briefly, 50 μl of a 50 μM ODN solution in 30 mM NaCl was injected, and elution was carried out in buffer containing 10 mM sodium phosphate, pH 6.9, 0.3 M NaCl at a flow rate of 0.6 ml/min. The HPLC elution fractions were divided into a high molecular weight aggregate portion (retention time 9-12.5 min) and a low molecular weight monomer portion (retention time 12.5-15 min). To address the association of ODN multimerization with immunostimulatory activity, the two fractions were collected, equal amounts were added to bone marrow-derived mononuclear cells (BDMC), and the culture supernatants were collected 48 h later for enzyme-linked immunosorbent assay, as described above.

For gel analysis, phosphorothioate and phosphodiester ODNs were mixed in RPMI 1640 with and without 2% tissue culture grade bovine serum albumin (Sigma) and incubated for 10 min at 37° C. 12 μl of the mixture were then separated on a 4-20% nondenaturing TBE polyacrylamide gel (Invitrogen). The oligonucleotides were visualized by staining with SYBR Green II (Molecular Probes, Eugene, Oreg.) under UV light. The protein bands were then detected with Coomassie Blue staining.

Circular Dichroism Spectroscopy—Oligonucleotides were resuspended in 10 mM sodium phosphate buffer, pH 7.2, containing 0.1 M KCl at a final concentration of 10 μM (final volume of 300 μl), boiled for 5 min, and annealed at 60° C. for 2 days. Dapic et al. (2002) Biochemistry 41:3676-3685. After slow cooling to room temperature, the samples were analyzed on an AVIV CD spectrometer (model 202, AVIV instruments, Inc., Lakewood, N.J.) using a wavelength scan from 320 to 200 nm at 25° C. Spectra were collected over three scans at 1-nm bandwidth, 1-nm wavelength step, and an average 0.5-s response time for each sample. Data are presented as the average of three scans with integrated curve fitting performed by Prism software (version 3.0; GraphPad Software, Inc., San Diego, Calif.).

Results

ODN Uptake Is Independent of TLR9—Phosphodiester ODNs with an average length of about 40 nucleotides that display improved cellular uptake compared with random sequence ODNs have been reported. Wu et al. (Jun. 10, 2003) Human Gene Therapy 14:849-860. Because the ODNs shown in Table 1 were selected for uptake by human B cells, it was necessary to confirm that they also effectively penetrated murine bone marrow-derived mononuclear cells. Experiments with fluorochrome-labeled ODNs showed that they were taken up 2-14-fold better than random sequence ODNs of the same length. In addition, bone marrow-derived cells from TLR9^{-/-} mice displayed an uptake efficiency similar to cells from wild type mice.

Sequence Requirements for Activation of Bone Marrow—derived Mononuclear Cells—To characterize the mechanisms involved in activation by these penetrating ODNs and to study the structural and functional requirements for stimulation, we carried out studies on murine bone marrow-derived mononuclear cells from different strains. A panel of ODNs containing different numbers of CpG dinucleotides and murine ISS motifs were first compared for their abilities to induce IL-12p^{40/}p70 secretion. As expected, ODNs without any CpG dinucleotides had no ISS activity (e.g., R10-5). The data are shown in Table 3. IL-12p40/p70 secretion is expressed as ng/ml.

TABLE 3

| | IL-12p10/p70 | | |
|---|---|---|---|
| ODN | BALB/c | (B6 × 129)F2 | TLR9^{-/-} |
| R10-5 | 0 | 0 | 0 |
| R10-9 | 0 | 0 | 0 |
| RI0-32 | 0.08 ± 0.04 | 0 | 0 |
| RI0-71 | 0.35 ± 0.08 | 0.22 ± 0.09 | 0 |
| RI0-13 | 0.29 ± 0.11 | 0.07 ± 0.02 | 0 |
| RI0-34 | 0.35 ± 0.04 | 0.06 ± 0.02 | 0 |
| RI0-60 | 10.45 ± 3.10 | 5.88 ± 1.23 | 0 |
| D-R15-8 | 1.76 ± 0.31 | 2.45 ± 0.60 | 0 |
| RI0-11 | 0.10 ± 0.02 | 0 | 0 |
| RI0-53 | 1.30 ± 0.26 | 0.38 ± 0.04 | 0 |
| RI0-86 | 0.44 ± 0.11 | 1.80 ± 0.94 | 0 |
| Controls | 0 | 0 | 0 |
| random 40 | | | |
| 1018 | 16.57 ± 4.01 | 1.04 ± 0.13 | 0 |
| LPS | 0.50 ± 0.19 | 0.39 ± 0.03 | 0.26 ± 0.04 |

Unexpectedly, however, no detectable IL-12 was released by cells treated with ODN R10-9, which contained the prototype ISS sequence motif AACGTT at the 5′ terminus. Cells stimulated with ODNs that contained at least two sets of CpG dinucleotides produced detectable levels of IL-12. Furthermore, phosphodiester ODN R10-60 showed comparable or even better IL-12 stimulation than the positive control ODN 1018, with a more nuclease-resistant phosphorothioate backbone. Table 3.

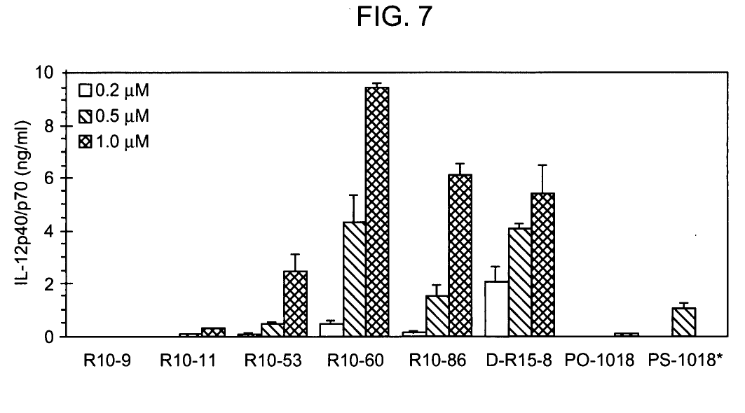

As little as 0.2 μM ODNs R10-53, R10-60, R10-86, and D-R15-8 were sufficient to induce detectable IL-12, and the levels increased in proportion to the ODN concentration, as shown in FIG. 7. In contrast, R10-9 was not able to elicit any IL-12 secretion at concentrations up to 1 μM. Together, these data demonstrated that phosphodiester ODNs can display equivalent immunostimulatory activity toward murine bone marrow-derived mononuclear cells as phosphorothioate ODN, and that a CpG motif is necessary but not sufficient for cell activation.

FIG. 7. Potency of selected phosphodiester ISS-ODNs. Ten-day-old bone marrow-derived mononuclear cells were incubated with the indicated ODNs (Table 2) or with the reference ISS-ODN 1018 in forms of phosphodiester (PO-1018) or phosphorothioate (PS1018 at 0.5 μM) at either 0.2, 0.5, or 1 μM for 2 days at 37° C. The cell media were harvested, and IL-12p40/p70 levels were determined by enzyme-linked immunosorbent assay. The results are means±S.D. of three replicates in a representative experiment. Similar results were obtained from at least two different experiments.

Role of the TLR-9 and MyD88 Pathways—Since there was no absolute correlation between an ISS motif and immunostimulatory activity among the selected ODNs, it was important to confirm that the ODNs signaled through the TLR9 and MyD88 pathway. No IL-12 production was observed from ODN-stimulated bone marrow-derived mononuclear cells from either TLR9^{-/-} or MyD88^{-/-} mice. Furthermore, the ODNs did not induce IκB kinase-β or Jun NH₂-terminal kinase activities in TLR9^{-/-} or MyD88^{-/-} cells, whereas bacterial lipopolysaccharide clearly activated these cells through the recently described MyD88-independent alternative pathway. The kinetics of cell activation revealed a maximum at 2 h after phosphodiester ODN application.

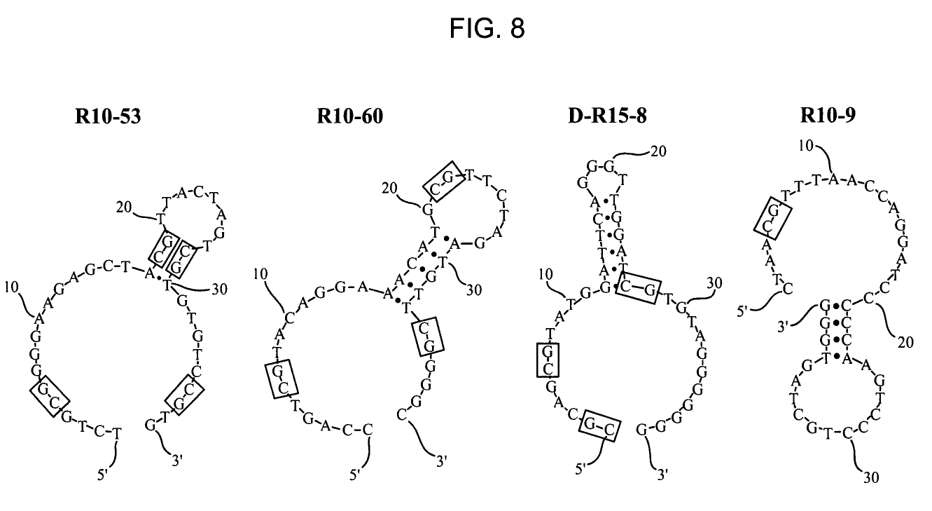

Association of Multimerization with ISS Activity—As the primary ISS motif (e.g. in R10-9) was insufficient for immunostimulatory activity, we evaluated whether a higher structure of an ODN also could influence its biologic properties. Results of the DNA mfold program showed that the CpG dinucleotide sequences in the active ISS-ODN, at their predicted lowest free energy states, were often in or near rigid stem loop structures, whereas the CpG in R10-9 was not, as shown in FIG. 8.

FIG. 8. A predicted structure derived from the DNA mfold program (available on the World Wide Web at bioinfo.rpi.edu/applications/mfold/old/dna. Sequences were submitted as linear DNA with folding at 37° C. and ionic conditions of 150 mM Na⁺, 0.5 mM Mg²⁺, oligomer type corrections. CpG dinucleotides were boxed.

The effect of various point mutations in the CpG located within predicted rigid loop structures of ISS-ODN on aggregate formation and ability to activate murine bone marrow derived mononuclear cells was analyzed. The nucleotide sequences of the oligodeoxynucleotides examined are presented in Table 4. CpG dinucleotides are in boldface type.

TABLE 4

| Name | Sequence |
|---|---|
| R10-53 | TCTGCGGGGAAGAGCTACGTTACTAGTCGTGTGTCCGTG; |
| | SEQ ID NO:13 |
| R10-53 | TCTGTGGGGAAGAGCTACGTTACTAGTCGTGTGTCCGTG; |
| (T5G) | SEQ ID NO:16 |
| R10-53 | TCTGCGGGGAAGAGCTATGTTACTAGTCGTGTGTCCGTG; |
| (T18G) | SEQ ID NO:17 |
| R10-53 | TCTGCGGGGAAGAGCTACGTTACTAGTTGTGTGTCCGTG; |
| (T28G) | SEQ ID NO:18 |
| R10-5 | TCTGCGGGGAAGAGCTACGTTACTAGTCGTGTGTCTGTG; |
| (T36G) | SEQ ID NO:19 |
| R10-60 | CCAGTCGTACAGGAAACATGCGTTCTAGATGTTCGGGGC; |
| | SEQ ID NO:10 |
| R10-60 | CCAGTTGTACAGGAAACATGCGTTCTAGATGTTCGGGGC; |
| (T6G) | SEQ ID NO:20 |
| R10-60 | CCAGTCGTACAGGAAACATGTGTTCTAGATGTTCGGGGC; |
| (T21G) | SEQ ID NO:21 |
| R10-60 | CCAGTCGTACAGGAAACATGCGTTCTAGATGTTTGGGGC; |
| (T34G) | SEQ ID NO:22 |
| R10-60a | CCAGTCGTACAGGAAACATGCGTTCTAGATGTTCG; |
| | SEQ ID NO:23 |
| R10-60b | CCAGTTGTACAGGAAACATGCGTTCTAGATGTTCG; |
| | SEQ ID NO:24 |
| D-R15-8 | CGCAGCGTATGGATTCAGGGTTGGATCGTGTAGGGGGGG; |
| | SEQ ID NO:11 |
| D-R15-8a | CGCAGCGTATGGATTCAGGGTTGGATCGTGTA; |
| | SEQ ID NO:25 |

Aggregate formation was determined by size exclusion HPLC analysis, and data are presented as percentage of peak area from two representative HPLC runs.

Immunostimulatory activity was determined based on IL-12p40/p70 production (pg/ml) from B6×129 F2 bone marrow-derived mouse mononuclear cells treated with 0.5 μM ODN for 2 days. Data are normalized relative to the corresponding parent ODN set at 100% activity. “mR10-60” is R10-60 with all CpG sites methylated. Similarly, “mD-R15-8” is D-R15-8 with all CpG sites methylated. Point mutations of the CpG located within the predicted rigid loop structures of ISS-ODN reduced their ability to activate murine bone marrow derived mononuclear cells. Table 5, R10-53 (T18G) and R10-60(T2 1 G).

TABLE 5

| | Length, | | | |
|---|---|---|---|---|
| Oligo | nt | CpG | Aggregates, % | Stimulation, % |
| R10-53 | 39 | 4 | 40, 46 | 100.0 ± 1.9 |
| R10-53(T5G) | 39 | 3 | 23, 26 | 92.6 ± 7.7 |
| R10-53(T18G) | 39 | 3 | 29, 32 | 5.3 ± 2.3 |
| R10-53(T28G) | 39 | 3 | 31, 31 | 40.0 ± 4.6 |
| R10-5(T36G) | 39 | 3 | 26, 27 | 70.8 ± 8.0 |
| R10-60 | 39 | 3 | 64, 65 | 100.0 ± 1.6 |
| R10-60(T6G) | 39 | 2 | 34, 36 | 52.2 ± 3.6 |
| R10-60(T21G) | 39 | 2 | 28, 35 | 24.2 ± 2.0 |
| R10-60(T34G) | 39 | 2 | 6, 19 | 71.4 ± 8.2 |
| R10-60a | 35 | 3 | 3, 6 | 29.2 ± 3.8 |
| R10-60b | 35 | 2 | 2, 3 | 2.9 ± 0.7 |
| mR10-60 | 39 | 0 | 26, 29 | 6.8 ± 1.239 |
| D-R15-8 | 39 | 3 | 71, 77 | 100.0 ± 12.7 |
| D-R15-8a | 32 | 3 | 1, 2 | 9.8 ± 4.0 |
| mD-R15-8 | 39 | 0 | 45, 48 | 9.3 ± 2.7 |

ODN aggregate formation was assessed by size exclusion HPLC. Samples were prepared at a concentration of 50 μM in 30 mM NaCl, and 50-μl aliquots were injected into the TSK column. High molecular weight aggregates eluted from the column starting at around 9 min, whereas monomers eluted at 13 min. Size exclusion HPLC analysis showed that the active ISS-ODN (R10-53, R10-60, and D-R15-8) formed multimers whereas the inactive ISS-ODN R10-9 did not. Nondenaturing polyacrylamide gel fractionation of the ODNs also confirmed the presence of multimers in the biologically active ODN samples. Removal of the guanine-rich sequences in the 3′-terminus of R10-60 and D-R15-8 (R10-60a and D-R15-8a) or near the 5′-end of R10-53 (R10-14Pu)) abolished aggregate formation.

Secondary structure formation was measured by CD spectroscopy. The CD analysis was carried out using a wavelength scan from 200 to 320 nm at 25° C. Spectra were collected from three scans at 1-nm bandwidth, wavelength step of 1 nm, and at an average time interval of 0.5 s. An integrated curve was derived from the average of three scans using Prism software. The presence of a parallel type guanine quartet is suggested by a positive maximum at ˜265 nm and a negative minimum near 240 nm. Circular dichroism spectroscopic analyses revealed absorption maxima that have been previously associated with the presence of guanine quartets, which are known to form aggregated structures.

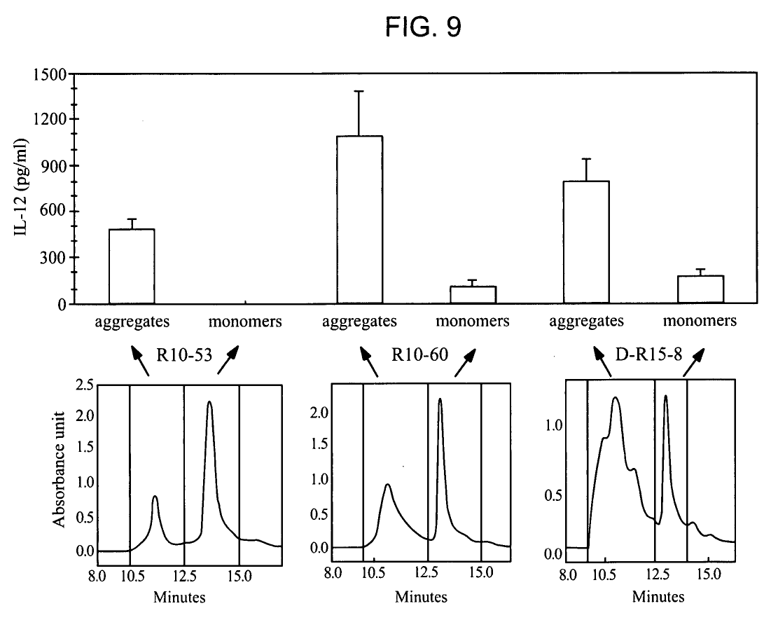

Although ODN multimerization correlated with enhanced immunostimulatory activity, this observation did not prove that aggregation was responsible for the stimulation potency. To address the question directly, ODN fractions of different sizes were collected from the HPLC elutes, and equal amounts were added to bone marrow-derived mononuclear cells. The maximal IL-12 was produced by cells that were stimulated with ODN aggregates, whereas at least 5-10-fold less IL-12 was observed from cells stimulated with ODN monomers, as shown in FIG. 9. Modified ISS-ODN, which had their 3′-guanine tails removed to diminish multimerization, also lost stimulation activity.

FIG. 9. ODN multimers are crucial for immunostimulatory activities. The multimeric and monomeric forms of the ISS-ODNs R10-53, R10-60, and D-R15-8 were collected from the HPLC eluates (bottom panel), diluted to equivalent A₂₆₀ concentrations in HPLC buffer, and sterilized through Spin-X columns. Aliquots of the separate fractions were added to bone marrow-derived mononuclear cells at a concentration of 1 μg/ml and incubated for 2 days, and the cell media were harvested to determine IL-12p40/p70 levels by enzyme-linked immunosorbent assay.

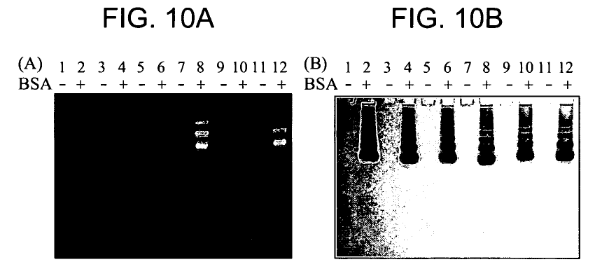

Under physiological conditions, phosphorothioate ODNs also aggregated and formed multimers by binding to plasma proteins, as shown in FIGS. 10A and 10B. Phosphodiester and phosphorothioate ODNs of the same sequence were incubated in medium with or without bovine serum albumin and analyzed by nondenaturing TBE PAGE. In the presence of bovine serum albumin, the prototype phosphorothioate ISS-ODNs, 1018 and 1826, were retained in the gel at higher molecular weights than the oligonucleotides in unsupplemented medium, as shown in FIG. 10A. The Coomassie Blue-stained bands suggested that these phosphorothioate ODNs co-migrated with the protein, as shown in FIG. 10B. In contrast, the phosphodiester counterparts of 1018 and 1826 were only visualized at the monomeric molecular weight and did not appreciably bind to protein. To determine whether ISS activity was retained in the absence of exogenous proteins, bone marrow-derived mononuclear cells were extensively washed to remove plasma proteins and then cultivated in completely serum/protein-free medium with the different ODNs.

FIGS. 10A and 10B. Protein aggregation of phosphorothioate ODNs. Phosphodiester R10-60 and phosphodiester (PO) and phosphorothioate (PS) prototype ISS-ODNs 1018 and 1826 (5′-TCCATGACGTTCCTGACGTT-3′; SEQ ID NO:26) were incubated in medium with 2% bovine serum albumin or without for 10 min at 37° C. and then separated by 4-20% TBE PAGE. The presence of ODNs was revealed by SYBR Green II staining (FIG. 10A) followed by Coomassie Blue staining for proteins (FIG. 10B). Lanes 1 and 2, medium; lanes 3 and 4, R10-60; lanes 5 and 6, PO-1018; lanes 7 and 8, PS-1018; lanes 9 and 10, PO-1826; lanes 11 and 12, PS-1 826.

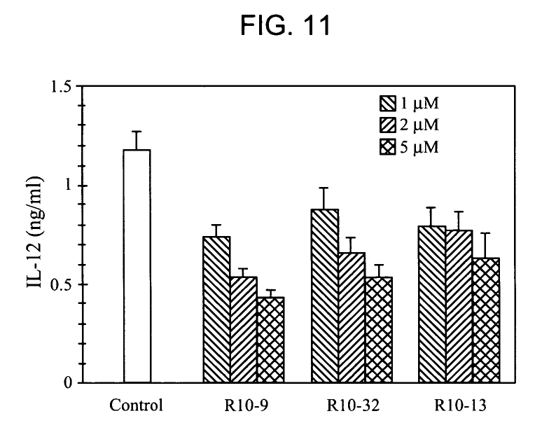

Biological Activities of ODN Monomers—The previous experiments demonstrated that monomeric ODNs with an ISS motif failed to effectively stimulate mouse bone marrow-derived mononuclear cells. However, the question remained whether the nonaggregating ODNs displayed other biological activities, such as the ability to antagonize cell activation by multimeric ISS-ODNs. To test this possibility, cells were stimulated with a mixture of R10-60 that forms aggregates and the nonaggregating ODN, R10-9, R10-32, or R10-13, at different ratios. A 10:1 molar excess of the monomeric ODNs reduced IL-12 production by approximately 64%, 55%, and 46%, respectively, and even a 2:1 ratio had significant inhibitory activity, as shown in FIG. 11. The latter concentrations of nonaggregating R10-9 were insufficient to impede R10-60 uptake.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A composition comprising lethally irradiated bacteria; and a pharmaceutically acceptable excipient, wherein the composition is formulated for mucosal delivery.

2. The composition of claim 1, wherein the composition is in a formulation suitable for oral delivery.

3. The composition according to claim 2, wherein the formulation is a liquid or gel formulation comprising an agent selected from a flavoring agent and a coloring agent.

4. The composition of claim 2, wherein the formulation is a solid formulation comprising a solid-based dry material.

5. The composition of claim 4, wherein the solid-based dry material is selected from a starch, gelatin, sucrose, dextrose, trehalose, and malto-dextrin.

6. The composition of claim 2, wherein said formulation is in the form of a lozenge, a capsule, a tablet, a liquid, or a gel.

7. The composition according to claim 1, wherein the pharmaceutically acceptable excipient is a food-grade excipient.

8. The composition of claim 7, wherein the food-grade carrier is selected from an edible oil, an emulsifier, a soluble fiber, a flavoring agent, a coloring agent, an edible fiber, and a sweetener.

9. The composition of claim 1, wherein the lethally irradiated bacteria are present at a concentration of from about 1×10² bacteria per dosage form to about 1×10¹² bacteria per dosage form.

10. The composition of claim 1, wherein the bacteria are generated by lethally irradiated live, pathogenic bacteria.

11. The composition of claim 1, wherein the live, pathogenic bacteria are selected from Salmonella enterica, Vibrio cholerae, Shigella sp., Mycobacterium tuberculosis, uropathogenic E. coli, enteropathogenic E. coli, Neisseria gonorrhea, Campylobacter jejuni, Brucella sp., Helicobacter pylori, Borrelia sp. and Francisella tularensis.

12. The composition of claim 1, further comprising a soluble antigen selected from a microbial antigen, a tumor antigen, and an allergen, wherein the antigen is in admixture with the bacteria.

13. The composition of claim 1, further comprising a killed or inactivated infectious micro-organism such as a virus.

14. The composition of claim 12, wherein the microbial antigen is selected from a viral antigen, a bacterial antigen, a fungal antigen, a protozoan antigen, a helminth antigen, or a mixture or two or more microbial antigens.

15. The composition of claim 12, wherein the allergen is selected from a plant allergen, an animal allergen, a fungal allergen, and a food allergen.

16. The composition of claim 12, wherein the bacteria are generated by lethally irradiating probiotic bacteria.

17. The composition of claim 1, wherein the bacteria are generated by lethally irradiating genetically modified bacteria, wherein, before irradiation, the genetically modified bacteria synthesize an exogenous antigen encoded by a recombinant expression vector.

18. The composition of claim 16, wherein the antigen is selected from microbial antigen, a tumor antigen, and an allergen, wherein the antigen is in admixture with the bacteria.

19. A method of inducing an immune response to an antigen in a mammalian subject, the method comprising administering to an individual a composition according to claim 1.

20. The method of claim 19, wherein an immune response to a live, pathogenic bacterium is induced.

21. The method of claim 19, wherein said composition is orally administered.

22. The method of claim 19, wherein said composition is administered by inhalation.

23. The method of claim 19, wherein said composition is nasally administered.

24. The method of claim 19, wherein said composition is rectally administered.

25. The method of claim 19, wherein said composition is vaginally administered.

26. A method of inducing an immune response to an antigen in a mammalian subject, the method comprising administering to an individual a composition according to claim 12.

27. A method of treating an allergic disorder in an individual, the method comprising administering to an individual sensitized to an allergen a composition according to claim 12, wherein the antigen is an allergen to which the individual is sensitized.
